(19) United States
(12) Patent Application Publication
(10) Pub. No.: US 2014/0301947 A1
(43) Pub. Date: Oct. 9, 2014

(54) METHODS TO PREDICT BINDING AFFINITY OF TSPO IMAGING AGENTS TO TSPO

(71) Applicants: David Owen, London (GB); Martin Wilkins, London (GB)

(72) Inventors: David Owen, London (GB); Martin Wilkins, London (GB)

(73) Assignee: IMPERIAL INNOVATIONS LIMITED, London (GB)

(21) Appl. No.: 14/124,606

(22) PCT Filed: May 31, 2012
(86) PCT No.: PCT/GB2012/051231

§ 371 Date: Apr. 8, 2014

Related U.S. Application Data
(60) Provisional application No. 61/493,738, filed on Jun. 6, 2011.
(60) Provisional application No. 61/527,221, filed on Aug. 25, 2011.

(51) Int. Cl. C12Q 1/68; A61K 51/04; A61K 49/00

(52) U.S. Cl. CPC C12Q 1/6883; A61K 49/0052; A61K 51/0459; A61K 51/0455
USPC 424/1.89; 435/6.11; 424/9.6; 514/263.3; 514/281; 424/1.81; 506/9

(57) ABSTRACT

A method for aiding in selecting or adjusting a dose of TSPO imaging or therapeutic agent for use with a subject, the method comprising the step of determining the subject's genotype for TSPO rs6971. A TSPO imaging or therapeutic agent for use in TSPO imaging or therapy in a subject, wherein the subject is a subject whose genotype at TSPO polymorphism position rs6971 is determined. The subject's genotype for TSPO may be taken into account when assessing the results of the TSPO imaging, for example normalising the subject's determined measure of binding of the TSPO agent to take account of the subject's determined genotype. The subject's genotype for TSPO may be taken into account when choosing the TSPO agent and/or the dose of TSPO agent. The subject may be a subject who has been determined to have genotype CC or CT at TSPO polymorphism position rs6971, for example when using a TSPO agent that binds with higher affinity to Aia147TSPO than to Thr147TSPO.

The present invention relates to the field of 18 kDa Translocator Protein TSPO (also called Peripheral Benzodiazepine Receptor) imaging methods and treatment methods.

The 18 kDa Translocator Protein TSPO is expressed within monocyte-derived cells and has been proposed as a marker of brain microglial activation (1). It is also a therapeutic target, for example in relation to anxiety disorders, for example as discussed in Owen et al (2011) Synapse 65, 257-259 (and references therein) and Pike et al (2011) J Med Chem 54, 366-373 (and references therein).

Quantitative imaging of TSPO with positron emission tomography (PET) has been technically challenging. The poor signal-to-noise ratio (SNR) of the first generation radioligand [¹¹C]PK11195 limits accurate quantification (2). Several second generation TSPO radioligands with improved SNR, including [¹¹C]PBR28, [¹⁸F]PBR06, [¹⁸F]FEPPA, [¹¹C]DAA1106, [¹¹C]DPA713 and [¹⁸F]PBR111, have been investigated in man (3).

However, there is substantial inter-subject variation in PET signal (4) and recent in vitro studies (5), (6) have revealed that this is due to inter-subject variation in the affinity of the second generation PET ligands for the TSPO. These radioligands appear to bind TSPO in brain tissue from different subjects in one of three ways: high affinity binders and low affinity binders (HABs and LABs) appear in radioligand binding studies to express a single binding site for TSPO with either high or low affinity respectively, whereas mixed affinity binders (MABs) appear to express broadly equal numbers of the HAB and LAB binding sites (5). However, it has not been confirmed that subjects fall neatly into one of these three categories. The fraction of HAB sites detected within MABs hitherto ranges from 38% to 83% (5). Given the difficulty in detecting two binding sites with radioligand binding studies when one binding site has much lower density than the other, such studies do not exclude the possibility that a continuum exists in expression of the HAB and LAB site. For example, subjects defined as HABs might in fact express the LAB binding site (and vice versa), but do so at a level which is below the threshold of detection (5).

Individuals display the same relative binding properties for all second generation radioligands (ie if an individual is a HAB with respect to PBR28 he/she is also a HAB with respect to the other second generation radioligands), although the exact Kᵢ values of the HAB and LAB binding sites vary between ligands (5). For example, PBR28 Kᵢ values for HABs and LABs are 4 nM and 200 nM respectively, whereas for PBR111 these values are 16 nM and 62 nM. Because the measured PET signal is a function of both the expression level of the target and the affinity of the radioligand for the target, subjects with identical TSPO expression will produce different PET signal if one is a HAB and one is a LAB. Prior knowledge of binding affinity therefore is required to make quantitative comparisons of TSPO expression between subjects in PET studies with these radioligands. This may not be necessary for the first generation radioligand, [¹¹C]PK11195, as in vitro studies with this ligand have not resolved separate HAB and LAB sites, suggesting that [¹¹C]PK11195 binds with very similar affinity in all subjects (6), which explains why low affinity binders have never been observed with this radioligand.

The mechanisms responsible for the different TSPO binding behaviours are not understood. Kreisl (2010) Neuroimage 49(4), 2924-2932 and Scarf (2011) J Nucl Med; 52, 677-680, for example, suggests various possible explanations. The TSPO exists both as a monomer and as part of a multimeric complex involving several TSPO monomers with associated proteins including the voltage dependant anion channel (VDAC) (1), (2), (3). Although subunit interactions are not necessary for drug ligands to bind the TSPO monomer (4), (5), binding of some drugs to the TSPO is influenced by these interactions (6). Hence the observed binding behaviour may be a function of the TSPO itself, the associated proteins, or the manner in which they interact to form the multimeric complex. We hypothesised that co-dominant expression of an underlying genetic polymorphism, in either the TSPO gene or in other genes encoding proteins in the TSPO complex, may be responsible for the described binding behaviour.

Here, we present the results of a genetic association study between TSPO agent binding affinity to human platelets and polymorphisms in genes encoding TSPO and associated proteins. We provide, for example, a simple genetic test for determination of TSPO binding class, which will, for example, contribute to quantitative interpretation of PET studies of TSPO expression.

A first aspect of the invention provides a method for aiding in selecting or adjusting a dose of TSPO imaging or therapeutic agent for use with a subject, the method comprising the step of determining the subject's genotype for TSPO rs6971.

A further aspect of the invention provides a TSPO imaging or therapeutic agent for use in TSPO imaging or therapy in a subject, wherein the dose of TSPO imaging or therapeutic agent is selected or adjusted taking into account the subject's genotype for TSPO rs6971.

A further aspect of the invention provides the use of a TSPO imaging or therapeutic agent in the manufacture of a medicament for use in TSPO imaging or therapy in a subject, wherein the dose of TSPO imaging or therapeutic agent is selected or adjusted taking into account the subject's genotype for TSPO rs6971.

As will be apparent to those skilled in the art, the subject may, as appropriate, be a subject in need of TSPO imaging or a subject in need of a TSPO therapeutic agent. The subject may alternatively be, for example, a healthy volunteer, for example as part of a clinical trial of TSPO imaging or therapy. The subject is typically a human subject.

As an example and as noted above, TSPO imaging may be useful in assessing brain inflammation or microglial activation, for example in a subject with or at risk of multiple sclerosis (MS), ischaemic stroke, herpes encephalitis, Parkinson's disease or Alzheimer's disease. As an example and as noted above, a TSPO therapeutic agent may be useful in treating an anxiety disorder or other neurological or psychiatric disorders, for example as discussed in Pike et al (2010) J Med Chem 54, 366-373 and references therein; and Rupprecht et al (2010) Nature Reviews Drug Discovery 9, 971-988.

TSPO is also potentially expressed outside the brain and may therefore be useful as a target for imaging or therapy in other tissues. Thus, imaging and therapy are not restricted to microglia in the brain, as TSPO is also expressed in other cells and used outside the brain, for example macrophages in atherosclerosis, vascular disease, inflammatory joint disease, cancer, lung disease. As TSPO imaging and therapy further improve (for example by making use of the present invention, which, for example, is considered to be useful in aiding analysis; and/or selection of appropriate subject; imaging/therapeutic agent and dose), TSPO imaging/therapy may be clinically useful in many more diseases. See, for example, the following references:
Atherosclerosis. 2008 November; 201(1):108-11. Epub 2008 Mar 14.
Increased peripheral benzodiazepine receptors in arterial plaque of patients with atherosclerosis: an autoradiographic study with [(3)H]PK 11195.
Fujimura Y, Hwang P M, Trout lii H, Kozloff L, Imaizumi M, Innis R B, Fujita M.
Detection and quantification of large-vessel inflammation with 11C-(R)-PK11195 PET/CT. Lamare F, Hinz R. Gaemperli O, Pugliese F, Mason J C, Spinks T, Camici P G, Rimoldi O E. J Nucl Med. 2011 January; 52(1):33-9. Epub 2010 Dec. 13
Peripheral-type benzodiazepine receptors in bronchoalveolar lavage cells of patients with interstitial lung disease. Branley H M, du Bois R M. Wells A U, Jones H A. Nucl Med Biol. 2007 July; 34(5):553-8. Epub 2007 Jun. 8.
PET scanning of macrophages in patients with scleroderma fibrosing alveolitis. Branley H M, du Bois R M, Wells A U, Jones H A. Nucl Med Biol. 2008 November; 35(8):901-9.
Quantification of (R)-[11C]PK11195 binding in rheumatoid arthritis. Kropholler M A, Boellaard R, Elzinga E H, van der Laken C J, Maruyama K, Kloet R W, Voskuyl A E, Dijkmans B A, Lammertsma A A. Eur J Nucl Med Mol Imaging. 2009 April; 36(4):624-31. Epub 2008 Nov. 29.
Noninvasive imaging of macrophages in rheumatoid synovitis using 11C-(R)-PK11195 and positron emission tomography. van der Laken C J, Elzinga E H, Kropholler M A, Molthoff C F, van der Heijden J W, Maruyama K, Boellaard R, Dijkmans B A, Lammertsma A A, Voskuyl A E. Arthritis Rheum. 2008 November; 58(11):3350-5.
Uptake of inflammatory cell marker [11C]PK11195 into mouse atherosclerotic plaques. Laitinen I, Marjamaki P, Nigren K, Laine V J, Wilson I, Leppanen P, Ylä-Herttuala S, Roivainen A, Knuuti J. Eur J Nucl Med Mol Imaging. 2009 January; 36(1):73-80. Epub 2008 Aug. 19.
Effect of PK11195, a peripheral benzodiazepine receptor agonist, on insulinoma cell death and insulin secretion. Park S Y, Cho N, Chang I, Chung J H, Min Y K, Lee M K, Kim K W, Kim S J, Lee M S. Apoptosis. 2005 May; 10(3):537-44.
Involvement of steroids in anti-inflammatory effects of PK11195 in a murine model of pleurisy. da Silva M B, Farges R C, Fröde T S. Mediators Inflamm. 2004 April; 13(2):93-103.
PK11195, a peripheral benzodiazepine receptor ligand, chemosensitizes acute myeloid leukemia cells to relevant therapeutic agents by more than one mechanism. Banker D E, Cooper J J, Fennell D A, Willman C L, Appelbaum F R, Cotter F E. Leuk Res. 2002 January; 26(1):91-106.

See also references below and in Example 4 relating to therapeutic uses of Vinpocetine, considered to be a TSPO therapeutic agent, as discussed below and in Example 4.

See also, for example US 2011/0070161 and WO 2010/109007.

The TSPO imaging agent or therapeutic agent is typically a TSPO imaging agent or therapeutic agent that binds with differential affinity to Ala147TSPO (encoded by the allele in which C is present at polymorphism RS6971) and Thr147TSPO (encoded by the allele in which T is present at polymorphism RS6971). This property is considered to be possessed by at least the great majority of the tested TSPO agents and is therefore considered to be possessed by at least the great majority of TSPO agents. PBR28 is considered to be an agent that binds with differential affinity to Ala147TSPO and Thr147TSPO, as are PBR06, DAA1106, Emapunil (also termed XBD173 when used as a drug or AC-5216 when used as a PET ligand), PBR111, DPA713, FEPPA, IGA-1 and the active enantiomer compound described in US 2011/0070161, as noted below, considered to be (4S)—N,N-diethyl-9-(2-fluoroethyl)-5-methoxy-2,3,4,4a,9,9a-hexahydro-1H-carbazole-4-carboxamide (also termed herein “(S)GE1”) (and, for example, related compounds or pharmaceutically acceptable salts or esters or enantiomers any thereof). Vinpocetine is also considered to be an agent that binds with differential affinity to Ala147TSPO and Thr147TSPO. It will be appreciated that the term TSPO imaging agent or therapeutic agent encompasses, for example, a non-radioactive precursor agent used in the preparation of the radioactive imaging agent, as will be apparent to those skilled in the art. It will further be appreciated that the term TSPO imaging agent or therapeutic agent encompasses agents that are considered to bind with high enough specificity and affinity to TSPO (for example either AlaTSPO or ThrTSPO or both considered together, for example in pooled results from tissue from multiple subjects) to be useful in imaging or therapy directed towards TSPO, as will be well known to those skilled in the art. It is envisaged that the TSPO imaging agent or therapeutic agent may bind to TSPO with a K_{d} lower than 300 nM. Examples of appropriate ranges of K_{d} values for TSPO binding for a potentially useful agent include 1 pM to 300 nM, 0.01 nM to 200 nM, 0.1 nM to 150 nM, 1 nM to 100 nM, 5 nM to 50 nM, and 10 nM to 25 nM (and, for the avoidance of doubt, combinations thereof).

TSPO agents for which affinity for Ala147TSPO and Thr147TSPO have separately been determined may be considered to fall into 1 of 6 broad categories: Class 1 (PBR28, PBR06, DAA1106. FEPPA) in general have very high affinity for the HAB site and a large difference in affinity between HABs and LABs, DAA1106 has very high affinity for the HAB site, but the difference between HABs and LABs is not as pronounced as might be expected.

Class 2 (emapunil) has large differences between HABs and LABs but not as great as class 1.

Class 3 and 4 (DPA713 and PBR111, which could be renamed 3a and 3b because they are so similar) have smaller differences in affinity between HAB and LAB site.

Class 5 (IGA-1, GE1 or (S)GE1 (the active enantiomer compound described in US 2011/0070161) has large differences between HABs and LABs but (like class 2) not as great as class 1. The Ki's are published in Pike et al (2011) J Med Chem 54, 366-373 as 1.57±1.03 nM (HAB); 1.82±11.09 nM (MAB) and 9.53±6.25 nM (LAB) for IGA-1. The Ki's for the racemic mixture (GE1) in which (S) GE1 is present are considered to be 12.35±2.1 nM (HAB) and 145.11±22.1 nM (LAB): see Example 3 below.

Class 6 (Vinpocetine) is considered to exhibit higher affinity binding in individuals otherwise classed as LABs than in individuals otherwise classed as HABs. Thus, vinpocetine is considered to have a higher affinity for Thr147TSPO than for Ala147TSPO.

The scaffolds of each class may be considered to be as set out below. Accordingly, TSPO agents corresponding to these scaffolds are considered to be examples of TSPO agents that bind with differential affinity to Ala147TSPO and Thr147TSPO.

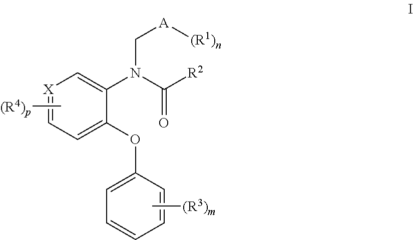

Wherein:
A is phenyl, N- or C-linked 5- or 6-membered aromatic or non-aromatic heterocyclic ring;

R¹ at each occurrence is independently selected from H, —OH, —NH₂, —CN, halogen, C₁₋₆alkyl-, C₁₋₃cycloalkyl-, C₁₋₆alkoxy-, C₁₋₆alkoxyC₁₋₆alkyl- and C₁₋₆alkoxyC₁₋₆halogen substituted alkyl-;
n is 1, 2 or 3;
R² is selected from H, —OH, —NH₂, —CN, halogen, C₁₋₆alkyl-, C₃₋₁cycloalkyl-, C₁₋₆alkoxy-, C₁₋₆alkoxyC₁₋₆alkyl- and C₁₋₆alkoxyC₁₋₆halogen substituted alkyl-;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
X is N, —CR⁴—, —CH—;

R⁴ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
p is 1, 2, 3 or 4

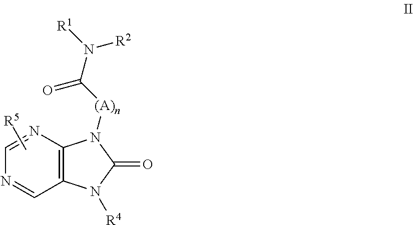

Wherein:
R¹ is selected from H, C₁₋₆alkyl or halogen-substituted C₁₋₆alkyl, C₃₋₇cycloalkyl- or halogen-substituted C₃₋₇cycloalkyl, C₁₋₄alkoxyC₁₋₆alkyl, halogen-substituted C₁₋₄alkoxyC₁₋₆alkyl;
R² is selected from H, C₁₋₆alkyl or halogen-substituted C₁₋₆alkyl, C₃₋₇cycloalkyl- or halogen-substituted C₃₋₇cycloalkyl, C₁₋₄alkoxyC₁₋₆alkyl, halogen-substituted C₁₋₄alkoxyC₁₋₆alkyl;
A is —C(═O)— or —CH₂—;
n is 0 or 1;
R³ at each occurrence is selected from —H, —OH, —NH₂, halogen, C₁₋₆alkyl-, C₃₋₇cycloalkyl-, C₁₋₆alkoxy- and C₁₋₆alkoxyC₁₋₆alkyl- and C₁₋₆alkoxyC₁₋₆halogen-substituted-alkyl; aryl and an N- or C-linked 5- or 6-membered aromatic ring;
m=1, 2 or 3
R⁴ is selected from H, C₁₋₆alkyl or halogen-substituted C₁₋₆alkyl, C₃₋₇cycloalkyl- or halogen-substituted C₃₋₇cycloalkyl, C₁₋₄alkoxyC₁₋₆alkyl or halogen-substituted C₁₋₄alkoxyC₁₋₆alkyl;
R⁵ at each occurrence is independently selected from H, —OH, —CN, halogen, —OR⁶, —NR⁶R⁷;
m is 1, 2, 3 or 4;
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-

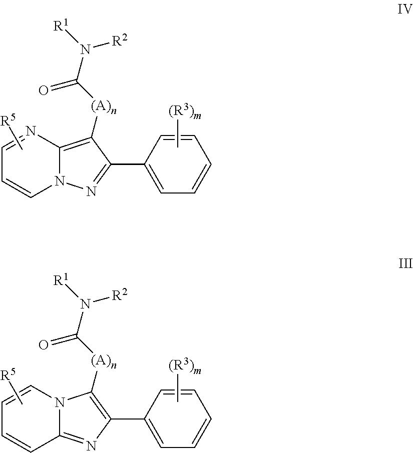

Wherein:
R¹ is selected from H, C₁₋₆alkyl or halogen-substituted C₁₋₆alkyl, C₃₋₇cycloalkyl- or halogen-substituted C₃₋₇cycloalkyl, C₁₋₄alkoxyC₁₋₆alkyl, halogen-substituted C₁₋₄alkoxyC₁₋₆alkyl;
R² is selected from H, C₁₋₆ alkyl or halogen-substituted C₁₋₆alkyl, C₃₋₇cycloalkyl- or halogen-substituted C₃₋₇cycloalkyl, C₁₋₄alkoxyC₁₋₆alkyl, halogen-substituted C₁₋₄alkoxyC₁₋₆alkyl;
A is —C(═O)— or —CH₂—;
n is 0 or 1;
R³ at each occurrence is selected from —H, —OH, —NH₂, halogen, C₁₋₆alkyl-, C₃₋₇cycloalkyl-, C₁₋₆alkoxy- and C₁₋₆alkoxyC₁₋₆alkyl- and C₁₋₆alkoxyC₁₋₆halogen-substituted-alkyl; aryl and an N- or C-linked 5- or 6-membered aromatic ring;
m=1, 2 or 3
R⁴ is selected from H, C₁₋₆alkyl or halogen-substituted C₁₋₆alkyl, C₃₋₇cycloalkyl- or halogen-substituted C₃₋₇cycloalkyl, C₁₋₄alkoxyC₁₋₆alkyl or halogen-substituted C₁₋₄alkoxyC₁₋₆alkyl;
R⁵ at each occurrence is independently selected from H, —OH, —CN, halogen, —OR⁶, —NR⁶R⁷;
m is 1, 2, 3 or 4;
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, =0, —NH₂, —CN, halogen, aryl and C₁₋₃alkoxy-

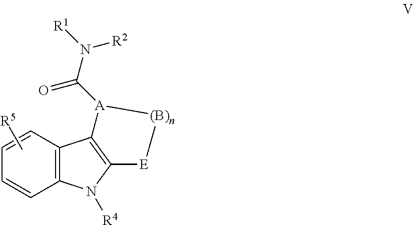

Wherein:
R¹ is selected from H, C₁₋₆ alkyl or halogen-substituted C₁₋₆alkyl, C₃₋₇cycloalkyl- or halogen-substituted C₃₋₇cycloalkyl, C₁₋₄alkoxyC₁₋₆alkyl, halogen-substituted C₁₋₄alkoxyC₁₋₆alkyl;
R² is selected from H, C₁₋₆ alkyl or halogen-substituted C₁₋₆alkyl, C₃₋₇cycloalkyl- or halogen-substituted C₃₋₇cycloalkyl, C₁₋₄alkoxyC₁₋₆alkyl, halogen-substituted C₁₋₄alkoxyC₁₋₆alkyl;
A is —C(═O)—, —CH— or —CH₂—;
B is alkyl
n is 0, 1 or 2
E is selected from alkyl, aryl and an N- or C-linked 5- or 6-membered aromatic ring for which substituent is R³;
R³ at each occurrence is independently selected from H, C₁₋₆ alkyl or halogen-substituted C₁₋₆alkyl, C₃₋₇cycloalkyl- or halogen-substituted C₃₋₇cycloalkyl, C₁₋₄alkoxyC₁₋₆alkyl, halogen-substituted C₁₋₄alkoxyC₁₋₆alkyl.
R⁴ is selected from H, C₁₋₆alkyl or halogen-substituted C₁₋₆alkyl, C₃₋₇cycloalkyl- or halogen-substituted C₃₋₇cycloalkyl, C₁₋₄alkoxyC₁₋₆alkyl or halogen-substituted C₁₋₄alkoxyC₁₋₆alkyl;
R⁵ at each occurrence is independently selected from H, —OH, —CN, halogen, —OR⁶, —NR⁶R⁷;
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, =0, —NH₂, —CN, halogen, aryl and C₁₋₃alkoxy-Vinpocetine (an example of Class 6) has the structure

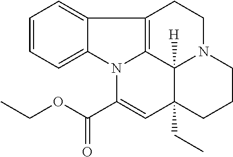

Vinpocetine (brand names: Cavinton, Intelectol; chemical name: ethyl apovincaminate) is a semisynthetic derivative alkaloid of vincamine (sometimes described as “a synthetic ethyl ester of apovincamine”),^{[1]} an extract from the periwinkle plant. 1.^{A} Lörincz C, Szász K, Kisfaludy L (1976). “The synthesis of ethyl apovincaminate”. Arzneimittel-Forschung 26 (10a): 1907. PMID 1037211.

Vinpocetine has been identified as binding the TSPO and is being used as a PET tracer at the Karolinksa Institute. See, for example, Gulyas B, Toth M, Vas A, Shchukin E, Kostulas K, Hillert J, Halldin C. Curr Radiopharm. 2012 Jan. 1; 5(1):19-28. Visualising neuroinflammation in post-stroke patients: a comparative PET study with the TSPO molecular imaging biomarkers [¹¹C]PK11195 and [¹¹C]vinpocetine.

Vinpocetine has been tested as a potential treatment for various diseases, including Alzheimer's Disease and stroke with no apparent success. In view of the present findings, it is possible that even if it causes dramatic improvements in the “low affinity binders” (in relation to PBR28 binding, for example), in which vinpocetine binds with high affinity but which make up only around 9% of the population, then the effect will have been masked by much lower, if any, effect in the rest of the population. See Example 4 for further discussion of Vinpocetine.

Further potentially suitable TSPO agents include those described in, for example, WO 2010/109007 and US 2011/0070161 (the compounds described therein are incorporated herein by reference), which describes enantiomers of a compound described in WO 2010/109007 and identifies the active enantiomer. The active enantiomer described in US 2011/0070161 is

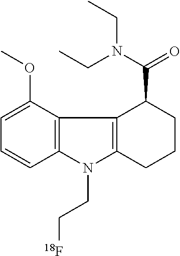 [SMILES: CCN(CC)C(=O)[C@H]1CCCc2c1c1c(OC)cccc1n2CC[18F]]

wherein the chiral centre has S configuration, and precursors thereof. This is considered to be (4S)—N,N-diethyl-9-(2-fluoroethyl)-5-methoxy-2,3,4,4a,9,9a-hexahydro-1H-carbazole-4-carboxamide, termed herein (S) GE1.

It will be understood that analogues and derivatives of the compounds described and referred to herein as potential TSPO agents are also intended to be included in the definition of TSPO agents.

Further potentially suitable TSPO agents include those described in Chauveau et al (2008) Eur. J. Nuc. Med. Mol. Imaging 35:2304-19, for example in Table 1, incorporated herein by reference. For example, suitable agents could include benzodiazepines, quinoline carboxamides (3-isoquinolinecarboxamides and quinoline-2-carboxamides), indoleacetamides, vinca alkaloids, oxodihydropurines, phenoxyarylacetamides or imidazopyridines and bioisteric structures (imidazopyridazines and pyrazolopyrimidines).

A further potentially suitable TSPO agent is SSR180575. See, for example Ferzaz B, Brault E, Bourliaud G, Robert J P, Poughon G, Claustre Y, Marguet F, Liere P, Schumacher M, Nowicki J P, Foumier J, Marabout B, Sevrin M, George P, Soubrie P, Benavides J, Scatton B. SSR180575 (7-chloro-N,N,5-trimethyl-4-oxo-3-phenyl-3,5-dihydro-4H-pyridazino[4,5-b]indole-1-acetamide), a peripheral benzodiazepine receptor ligand, promotes neuronal survival and repair. Journal of Pharmacology and Experimental Therapeutics. 2002 June; 301(3):1067-78.

An agent that binds with differential affinity to Ala147TPSO and Thr147TSPO may typically have a Ki for the lower affinity binding (typically binding with Thr147TSPO) that is at least 1.2, 1.5, 2, 2.5, 3, preferably at least 4, 4.5, 5, 6, 7, 8, 10, 12, 15 or 20-fold higher than the Ki for the higher affinity binding (typically binding with Ala147TSPO).

The TSPO imaging or therapeutic agents mentioned above will be well known to those skilled in the art. They are also discussed further in, for example, the following, and references therein: Owen et al (2010) J Cer Blood Flow Metab 30, 1608-1618; Owen et al (2011) J Nucl Med 52, 24-32; Owen et al (2011) Synapse 65, 257-259; Pike et al (2010) J Med Chem 54, 366-373 Evaluation of Novel N(1)-Methyl-2-phenylindol-3-ylglyoxylamides as a New Chemotype of 18 kDa Translocator Protein-Selective Ligand Suitable for the Development of Positron Emission Tomography Radioligands. IGA-1 refers to compound 31 of Pike et al (2010) supra. Pike et al (2010) supra, for example, also refers to further TSPO imaging or therapeutic agents.

See also, for example: JP 2000001476 A, WO 9906353 (for example DAA1106); WO 2008022396 (for example DPA713/PBR111:); WO 9709308 (for example IGA-1 (indolyl family)): JP 2009132701, JP 2008031093, WO 2006096435, US 20060199805, WO 2006093041, WO 2002010167, JP 2001048882, WO 9928320 (for example Emapunil).

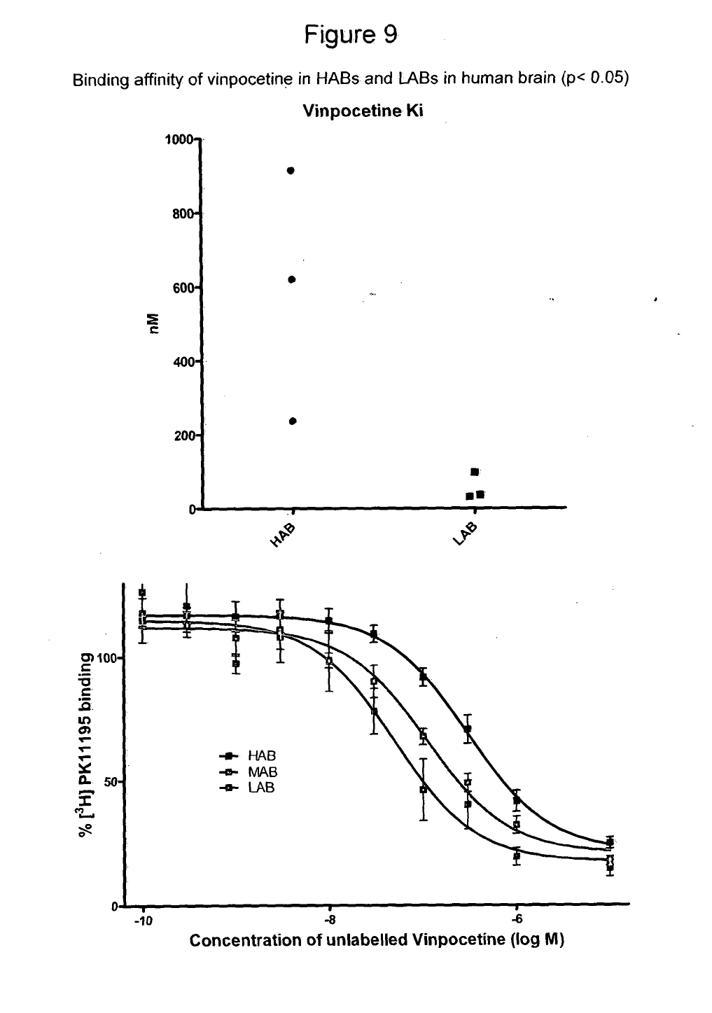

Example 2 and FIG. 3, for example, show structures and Ki's determined for some TSPO imaging and therapeutic agents. FIG. 9 shows vinpocetine Ki's.

The Ki's may be Ki's determined using any suitable binding assay, as will be apparent to those skilled in the art. Examples of suitable methods are described in Owen et al (2011) J Nucl Med 52, 24-32.

Thus, for example, the binding affinity of the compound may be determined in tissue derived from a HAB and in tissue derived from a LAB. An appropriate method may comprise the following steps:
obtain samples from human volunteers of tissue which contains TSPO (for example brain (for example for archive samples) or platelets (for example from blood)). Examples of suitable samples are described in, for example Owen et al (2011) supra. Thus, for example, a membrane preparation from brain or platelet tissue may be used, as appropriate, for example as described in Owen et al (2011) J Nucl Med 52, 24-32
Characterize each subject as HAB, LAB or MAB using a radioligand binding assay, typically with PBR28. PBR28 is useful to characterize tissue because affinity differences with PBR28 are large. Alternatively, a subject may be classified as a HAB, MAB or LAB based on genotype at position TSPO rs6971 using any tissue suitable for genotypic analysis, for example a blood sample, as will be well known to those skilled in the art.
Once subjects have been classified into HABS, MABS and LABS, measure the affinity of the experimental molecule for the TSPO, typically using only those subjects classified as HAB or LAB.
Typically perform a statistical test to ascertain whether there are significant differences in affinity for the experimental compound when comparing HABs and the LABs.
The Ki values can then be used as described herein, for example to correct PET data or guide drug dosing using standard dose occupancy equations.

Affinity may be measured using known techniques, for example using standard radioligand binding assays (well known to those skilled in the art) such as with a saturation or competition assay:

### 1) Saturation Assay

The compound of interest is radiolabelled: tissue is incubated in increasing concentrations of radiolabelled radioligand, and specific binding (ie amount of radiolabelled ligand bound to TSPO) is measured at each concentration. A plot of ligand concentration (x axis) and specific binding (y axis) is generated. This curve plateaus at y=total amount of TSPO present in the sample (Bₘₐₓ). The Kd is the concentration of radioligand required to reach 50% of the Bₘₐₓ.

### 2) Competition Assay

Because radiolabelling a compound is very expensive, it may be easier to take a commercially available radiolabelled TSPO targeting agent (for example [³H]PK11195) and use the agent of interest (which may be the same agent as the labelled TSPO targeting agent) to displace it. As increasing concentrations of unlabelled agent is added, the amount of [³H]PK11195 (for example) bound to the TSPO decreases. The amount of non labeled agent added (x axis) is plotted against residual [³H]PK11195 binding (y axis). The concentration of unlabelled drug required to displace 50% of the [³H]PK11195 is calculated from the graph. This value (called an IC50) is converted to a Ki by a simple equation which takes into account the Kd of [³H]PK11195 and the concentration of [³H]PK11195 used in the experiment.

The IC50 may be calculated by, as appropriate, determining from the graph the concentration of unlabelled drug required to displace 50% of the displaceable [³H]PK11195 (for example) or the concentration of unlabelled drug required to displace 50% of the total [³H]PK11195 (for example), as will be apparent to those skilled in the art.

If the agent of interest, when present in excess, for example at a concentration of 3 mM, does not displace at least 70% of the labelled TSPO targeting agent (reference agent), for example [³H]PK11195, then it may be more appropriate to determine the Kd of the agent of interest by radiolabelling the agent with, for example, [³H], and performing a saturation assay.

In more detail, as an example, a 48 well plate may be used, for example with 8-12 different concentrations of experimental drug for each experiment (be it the radiolabelled drug for saturation studies or the un-radiolabelled drug for competition studies). Concentrations may range from around 0.1 nM, increasing in roughly half logs (ie 0.1 nM, 0.3 nM, 1 nM, 3 nM etc) up to around 10,000 nM. 3-4 wells may be used for each concentration and the average taken to increase the precision around the estimate for each data point. The amount of tissue material required depends on how much TSPO is in the chosen tissue. For brain ˜100 micrograms of protein in each well (500 microlitres) may typically be used, giving a protein concentration of ˜200 micrograms/ml. Similar amounts may be used for platelets. For a tissue with very high TSPO levels (eg adrenal gland) a lot less protein would be needed. For competition assays, the amount of radiolabelled PK11195 which goes into each well may typically be 5 nM (Radioactive concentration ˜1.0 mCi/ml). Typically five HAB samples of HAB and five LAB samples may be used ie n=5 HAB and n=5 LAB (see the Examples) but other sample numbers may also be used.

Ki (from a competition assay) and Kd (from a saturation assay) can generally be treated as essentially equivalent.

As will be appreciated by those skilled in the art, a more accurate result may be obtained by steps such as increasing the sample size; using more wells per concentration of drug; using more concentrations; repeating the test and averaging the results etc. Taking one or more of such steps may reveal a small but statistically significant difference between binding affinities for a particular agent for Ala147TSPO and Thr147TSPO. A small but statistically significant difference may be clinically significant, as described herein. Typically, however, relevant differences in affinity will be readily apparent, for example where the Ki's differ by at least 1.5, 2, 2.5, 3, preferably at least 4, 4.5, 5, 6, 7, 8, 10, 12, 15 or 20-fold.

PK11195, for example [³H]PK11195 may not bind with significant differential affinity to Ala147TPSO and Thr147TSPO. No significant difference in binding affinity has been detected with PK11195 when performing the assay as described in Owen et al (2011) J Nucl Med 52, 24-32, though it is possible that there is a small difference that has not been detected. Should a more accurate assay find a significant difference (for example ˜20% ie a ratio of 1.2) then PK1195 imaging may also be improved by knowing the genotype, as described herein.

Typically, for an agent that binds with higher affinity to Ala147TSPO (encoded by the allele in which C is present at polymorphism rs6971) than to Thr147TSPO (encoded by the allele in which T is present at polymorphism rs6971), when the subject's genotype for TSPO rs6971 is determined to be CC the dose of TSPO imaging or therapeutic agent may be reduced (for example relative to a standard dose of TSPO imaging or therapeutic agent; or relative to a dose that may be selected in the absence of information of TSPO genotype or binding affinity for that subject). Typically, for an agent that binds with higher affinity to Ala147TSPO than to Thr147TSPO, when the subject's genotype for TSPO rs6871 is determined to be CT, the dose of TSPO imaging or therapeutic agent may be increased (for example relative to a standard dose of TSPO imaging or therapeutic agent; or relative to a dose that may be selected in the absence of information of TSPO genotype or binding affinity for that subject). Typically, for an agent that binds with higher affinity to Ala147TSPO than to Thr147TSPO, when the subject's genotype for TSPO rs6871 is determined to be TT, the subject may be identified as unsuitable for TSPO imaging or therapy with such an agent; or alternatively the dose of TSPO imaging or therapeutic agent may be increased (for example relative to a standard dose of TSPO imaging or therapeutic agent; or relative to a dose that may be selected in the absence of information of TSPO genotype or binding affinity for that subject) to a greater amount than if the subject's genotype for TSPO rs6871 had been determined to be CT.

For an agent (for example vinpocetine) that binds with higher affinity to Thr147TSPO (encoded by the allele in which T is present at polymorphism rs6971) than to Ala147TSPO (encoded by the allele in which C is present at polymorphism rs6971), when the subject's genotype for TSPO rs6971 is determined to be TT the dose of such a TSPO imaging or therapeutic agent may be reduced (for example relative to a standard dose of TSPO imaging or therapeutic agent; or relative to a dose that may be selected in the absence of information of TSPO genotype or binding affinity for that subject). When the subject's genotype for TSPO rs6871 is determined to be CT, the dose of such a TSPO imaging or therapeutic agent may be increased (for example relative to a standard dose of TSPO imaging or therapeutic agent; or relative to a dose that may be selected in the absence of information of TSPO genotype or binding affinity for that subject). When the subject's genotype for TSPO rs6871 is determined to be CC, the subject may be identified as unsuitable for TSPO imaging or therapy with such an agent; or alternatively the dose of the TSPO imaging or therapeutic agent may be increased (for example relative to a standard dose of TSPO imaging or therapeutic agent; or relative to a dose that may be selected in the absence of information of TSPO genotype or binding affinity for that subject) to a greater amount than if the subject's genotype for TSPO rs6871 had been determined to be CT.

The ratio of specific signal for HABs, MABs and LABs with some TSPO ligands is shown below.

| Ligand | LAB | MAB | HAB |
|---|---|---|---|
| PBR28 | 1 | 28.2 | 55.3 |
| PBR06 | 1 | 9.2 | 17.3 |
| DAA1106 | 1 | 2.9 | 4.7 |
| PBR111 | 1 | 2.5 | 4.0 |
| DPA173 | 1 | 2.7 | 4.4 |
| GE1 (racemic) | 1 | 6.4 | 11.8 |
| PK11195 | 1 | 0.9 | 0.8 |

Thus, for PBR06 (as an example), the dose selected for a LAB (low affinity binding) subject (with genotype TT) may be higher than the dose for a HAB (high affinity binding) subject (with genotype CC) or the dose for a MAB (mixed affinity binding) subject (with genotype CT).

It will be appreciated that the dose of a PET ligand may be limited by the acceptable dose of radioactivity. Typically, the dose may be chosen to be near the maximum, because typically the more radioactivity the better quality the data derived from the scan. So there may be limited scope for adjusting the dose depending on the genotype. Nevertheless, it may still be possible and desirable to adjust the dose. There may be more scope for altering the dose for a therapeutic agent or other non-radioactive agent.

To achieve (where possible) the same % of TSPO that is occupied by the drug, the required free concentration of the drug in the tissue can be worked out by standard dose occupancy equations, for example:

% of binding sites occupied by drug (occupancy)=Dose of drug/(dose of drug+Kd of drug)

This can be rearranged to

Dose of drug=(occupancy×Kd)/(1−occupancy)

For a given occupancy that is required, the dose needed is proportional to the Kd, and if there is a 17 fold difference in Kd there would theoretically need to be a 17 fold difference in dose assuming that all other factors remain equal. In practice there may be differences between people in other ways, for example how they metabolise the drug, so the calculation provides guidance rather than on its own indicating the dose needed.

A further aspect of the invention provides a method for TSPO imaging or therapy wherein the subject's genotype at TSPO polymorphism position rs6971 is determined. The subject's genotype for TSPO may, for example, be taken into account when assessing the results of the TSPO imaging. As a further example, the subject's genotype for TSPO may alternatively or in addition be taken into account when choosing the TSPO agent and/or the dose of TSPO agent (for example as discussed above).

A further aspect of the invention provides a TSPO imaging or therapeutic agent for use in TSPO imaging or therapy in a subject, wherein the subject is a subject whose genotype at TSPO polymorphism position rs6971 is determined. The subject's genotype for TSPO may, for example, be taken into account when assessing the results of the TSPO imaging. As a further example, the subject's genotype for TSPO may alternatively or in addition be taken into account when choosing the TSPO agent and/or the dose of TSPO agent (for example as discussed above).

A further aspect of the invention provides the use of a TSPO imaging or therapeutic agent in the manufacture of a medicament for use in TSPO imaging or therapy in a subject, wherein the subject is a subject whose genotype at TSPO polymorphism position rs6971 is determined. The subject's genotype for TSPO may, for example, be taken into account when assessing the results of the TSPO imaging. As a further example, the subject's genotype for TSPO may alternatively or in addition be taken into account when choosing the TSPO agent and/or the dose of TSPO agent (for example as discussed above).

When a subject is scanned in PET, a binding potential (BP) or volume of distribution (Vd) for a region of interest is calculated. These are two slightly different ways of attempting to characterise how much radioligand is bound specifically to the target in the region of interest. Once the BP or Vd has been calculated, it can be corrected for binding class by, for example, scaling up the values obtained in a MAB subject or a LAB subject to that of a HAB subject. Eg, with PBR06, the value obtained in a LAB subject would be multiplied by 17 (see table above) and the value obtained in a MAB subject by about 2. Some further corrections will be required in order to take into account other factors such as non specific binding, as will be apparent to those skilled in the art.

Thus, the method (or TSPO agent or use, as appropriate) may comprise normalising the subject's determined measure of binding of the TSPO agent to take account of the subject's determined genotype.

The choice of agent may comprise considering the affinity of potential agents for the subject's form of TSPO as revealed by the subject's genotype at TSPO polymorphism position rs6971, and selecting for further consideration an agent that has acceptable affinity for the subject's form of TSPO. For example, it is considered that PBR28 may have too low affinity for Thr147TSPO to be useful (as a radioactive imaging agent) in a subject who has only this form of TSPO (ie is homozygous for Thr147TSPO).

The affinity that is acceptable will depend upon several factors: for example for a radioactive imaging agent a relatively higher affinity may be required than for a non-radioactive therapeutic agent with no significant side effects, where a relatively lower affinity may be acceptable.

Alternatively or in addition, the subject may be a subject who has been determined to have genotype CC or CT at TSPO polymorphism position rs6971, for example when using a TSPO agent that binds with higher affinity to Ala147TSPO (encoded by the allele in which C is present at polymorphism rs6971) than to Thr147TSPO (encoded by the allele in which T is present at polymorphism rs6971), for example when using an agent where the affinity for Thr147TSPO is too low for a useful signal to be obtained. For example, the affinity of PBR28 for Thr147TSPO may be too low for a useful signal to be obtained using this agent in a subject who has genotype TT at TSPO polymorphism position rs6971.

As a further alternative or addition, the subject may be a subject who has been determined to have genotype TT or CT at TSPO polymorphism position rs6971, for example when using a TSPO agent that binds with higher affinity to Thr147TSPO (encoded by the allele in which T is present at polymorphism rs6971) than to Ala147TSPO (encoded by the allele in which C is present at polymorphism rs6971)), for example when using an agent where the affinity for Ala147TSPO is too low for a useful signal to be obtained. Vinpocetine is considered to be a TSPO agent that binds with higher affinity to Thr147TSPO than to Ala147TSPO.

As a further alternative or addition, the agent may be an agent that has been chosen taking into account the affinity of potential agents for the subject's form of TSPO as revealed by the subject's genotype at TSPO polymorphism position rs6971, typically an agent selected for further consideration as having an acceptable affinity for the subject's form of TSPO, as discussed above.

A further aspect of the invention provides a method for TSPO imaging or therapy using a TSPO imaging or therapeutic agent wherein the method is performed on a subject who has been determined to have genotype CC or CT at TSPO polymorphism position rs6971, for example when the TSPO imaging or therapeutic agent is a TSPO agent that binds with higher affinity to Ala147TSPO (encoded by the allele in which C is present at polymorphism rs6971) than to Thr147TSPO (encoded by the allele in which T is present at polymorphism rs6971), for example an agent where the affinity for Thr147TSPO is too low for a useful signal to be obtained (eg with that agent in a subject with Thr147TSPO only).

For example, as noted above, the affinity of PBR28 for Thr147TSPO may be too low for a useful signal to be obtained using this agent in a subject who has genotype TT at TSPO polymorphism position rs6971. TSPO imaging or therapy may be decided against for a subject whose genotype at TSPO polymorphism position rs6971 is determined to be TT when using such an agent.

Thus, when using such an agent, for example PBR28, the subject may be selected for TSPO imaging or therapy only if they have genotype CC or genotype CT at TSPO polymorphism position rs6971; or only if they have genotype CC at TSPO polymorphism position rs6971, which is considered the genotype most suitable for TSPO imaging or therapy using such an agent, for example PBR28.

A further aspect of the invention provides a method for aiding in determining whether a subject is suitable for TSPO imaging or therapy using a TSPO imaging or therapeutic agent, the method comprising the step of selecting the subject as suitable for TSPO imaging or therapy based on the subject's genotype at TSPO polymorphism position rs6971. Thus, a further aspect of the invention provides a method for aiding in determining whether a subject is suitable for TSPO imaging or therapy using a TSPO imaging or therapeutic agent, the method comprising the step of selecting the subject as suitable for TSPO imaging or therapy if the subject has genotype CC or CT at TSPO polymorphism position rs6971; or alternatively if they have genotype CC at TSPO polymorphism position rs6971, which is considered the genotype most suitable for TSPO imaging or therapy, for example, in both case, when the TSPO imaging or therapeutic agent is a TSPO agent that binds with higher affinity to Ala147TSPO (encoded by the allele in which C is present at polymorphism rs6971) than to Thr147TSPO (encoded by the allele in which T is present at polymorphism rs6971), for example an agent where the affinity for Thr147TSPO is too low for a useful signal to be obtained (eg with that agent in a subject with Thr147TSPO only).

A further aspect of the invention provides a method for TSPO imaging or therapy using a TSPO imaging or therapeutic agent wherein the method is performed on a subject who has been determined to have genotype CC or CT at TSPO polymorphism position rs6971, for example when the TSPO imaging or therapeutic agent is a TSPO agent that binds with higher affinity to Ala147TSPO (encoded by the allele in which C is present at polymorphism rs6971) than to Thr147TSPO (encoded by the allele in which T is present at polymorphism rs6971), for example an agent where the affinity for Thr147TSPO is too low for a useful signal to be obtained. For example, the affinity of PBR28 for Thr147TSPO may be too low for a useful signal to be obtained using this agent in a subject who has genotype TT at TSPO polymorphism position rs6971. TSPO imaging or therapy may be decided against for a subject whose genotype at TSPO polymorphism position rs6971 is determined to be TT when using such an agent.

A further aspect of the invention provides a method for aiding in determining whether a subject is suitable for TSPO imaging or therapy using a TSPO imaging or therapeutic agent, the method comprising the step of selecting the subject as suitable for TSPO imaging or therapy if the subject has genotype TT or CT at TSPO polymorphism position rs6971; or alternatively if they have genotype TT at TSPO polymorphism position rs6971, which is considered the genotype most suitable for TSPO imaging or therapy, for example, in both case, when the TSPO imaging or therapeutic agent is a TSPO agent that binds with higher affinity to Thr147TSPO (encoded by the allele in which T is present at polymorphism rs6971) than to Ala147TSPO (encoded by the allele in which C is present at polymorphism rs6971), for example an agent where the affinity for Ala147TSPO is too low for a useful signal to be obtained (eg with that agent in a subject with Ala147TSPO only).

A further aspect of the invention provides a method for TSPO imaging or therapy using a TSPO imaging or therapeutic agent wherein the method is performed on a subject who has been determined to have genotype TT or CT at TSPO polymorphism position rs6971, for example when the TSPO imaging or therapeutic agent is a TSPO agent that binds with higher affinity to Thr147TSPO (encoded by the allele in which T is present at polymorphism rs6971) than to Ala147TSPO (encoded by the allele in which C is present at polymorphism rs6971), for example an agent where the affinity for Ala147TSPO is too low for a useful signal to be obtained. TSPO imaging or therapy may be decided against for a subject whose genotype at TSPO polymorphism position rs6971 is determined to be CC when using such an agent.

The subject's genotype may be assessed at the time of considering or carrying out the TSPO imaging or therapy; or it may be assessed separately, for example as part of a wider genetic characterisation of the subject. The subject's characterisation may then be stored; and accessed when considering or carrying out the TSPO imaging or therapy on the subject. The genotyping may be done on any suitable tissue from the subject, for example on a blood sample from the subject, as will be well known to those skilled in the art. The genotyping may be done by any suitable technique for determining genotype, as will be well known to those skilled in the art and as discussed further below.

A further aspect of the invention provides a reagent specifically for assessing a subject's genotype at TSPO rs6971 for use in a method of TSPO imaging or therapy.

A further aspect of the invention provides the use of a reagent specifically for assessing a subject's genotype at TSPO rs6971 in the manufacture of a medicament (ie a diagnostic reagent) for use in a method of TSPO imaging or therapy.

The reagent specifically for assessing a subject's genotype at TSPO rs6971 is typically a nucleic acid molecule that hybridises specifically to TSPO nucleic acid and is useful in distinguishing between the possible alleles at TSPO rs6971. Many different techniques are available by which genotype may be determined, some examples of which are mentioned or described in Example 1, for example in Table 2. Exemplary techniques include techniques based on PCR or FRET. For example, the TaqMan® (Applied Biosystems, CA, USA)), Luminex-based Flow Assorted SNP Typing (FAST) (7) procedure may be used.

Information on the TSPO gene and on rs6971 may be found at, for example, http://www.genecards.org/cgi-bin/carddisp.pl?gene=TSPO. The rs6971 polymorphism is GGTCGC/TGAAGG, as will be apparent to those skilled in the art.

As a further example, genotype at TSPO rs6971 may be determined using a PCR-restriction fragment length polymorphism method. Suitable forward and reverse primers may be:

5′-TGGGACAGGCACTTGGGTGAAC-3′(forward)
5′-AAGCGTGACGGCCACCACATCA-3′(reverse)

The PCR products from this amplification are digested with enzyme NruI, and, for example, separated by electrophoresis on 2.5% Ultrapure agarose-Tris-borate EDTA geles (Life Technologies, Inc., Gaithersburg, Md.).

A further aspect of the invention provides a kit of parts comprising a TSPO imaging or therapeutic agent and a reagent specifically for assessing a subject's genotype at TSPO rs6971.

It will be appreciated that assessment of a subject's phenotype for TSPO, for example through assessing whether a subject has high affinity, low affinity, or mixed affinity binding for a TSPO imaging or therapeutic agent is not assessment of the subject's genotype at TSPO rs6971.

The listing or discussion of an apparently prior-published document in this specification should not necessarily be taken as an acknowledgement that the document is part of the state of the art or is common general knowledge.

Any document referred to herein is hereby incorporated by reference.

The invention is now described in more detail by reference to the following, non-limiting, Figures and Examples.

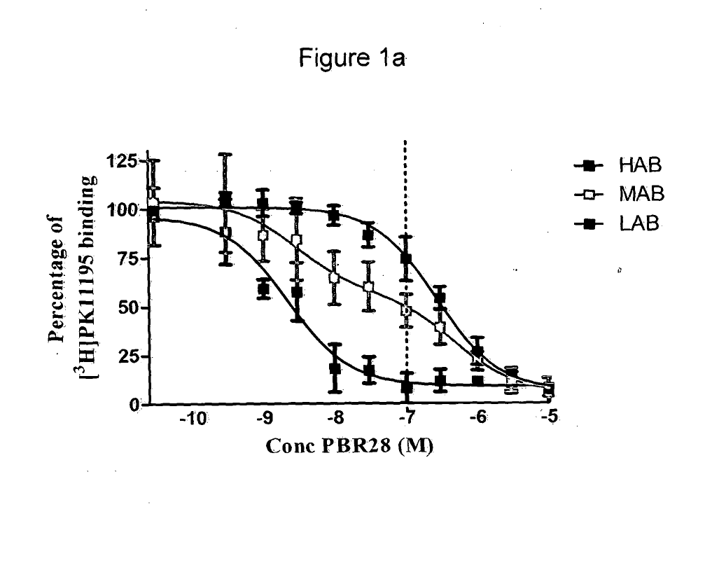

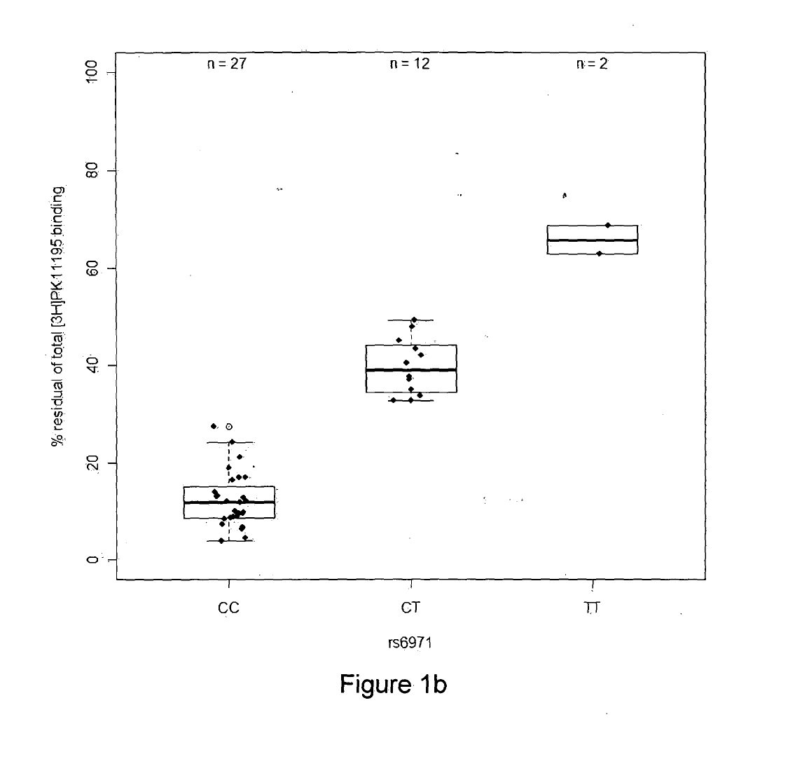

FIG. 1

(a) Competition binding assay using unlabelled PBR28 to displace [³H]PK11195, in human platelets isolated from whole blood (n=41). The dashed vertical line indicates the concentration of PBR28 used to generate FIG. 1b.

(b) Box-whisker plot of the residual [³H]PK11195 binding in the presence of 100 nM unlabelled PBR28 (expressed as a percentage of the total [³H]PK11195 binding in the absence of PBR28) stratified on rs6971 genotype. Percentage residual of total binding is plotted as blue diamonds for each individual. The outlier in the CC genotype group, as indicated by the open dot of the box-whisker plot, is also represented by the adjacent blue diamond.

Abbreviations:

HABs; High affinity binders.

LABs; Low affinity binders.

MABs; Mixed affinity binders.

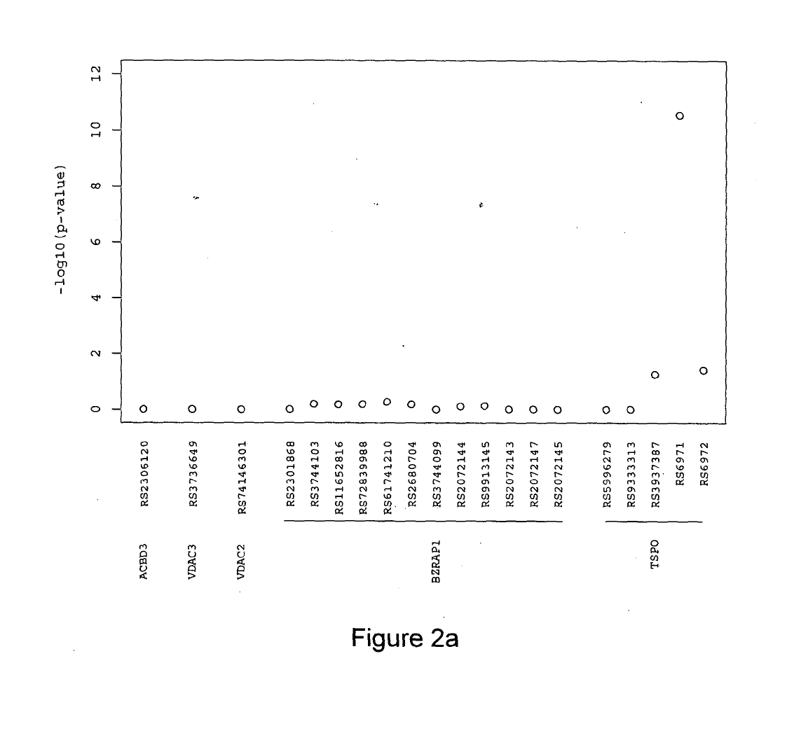

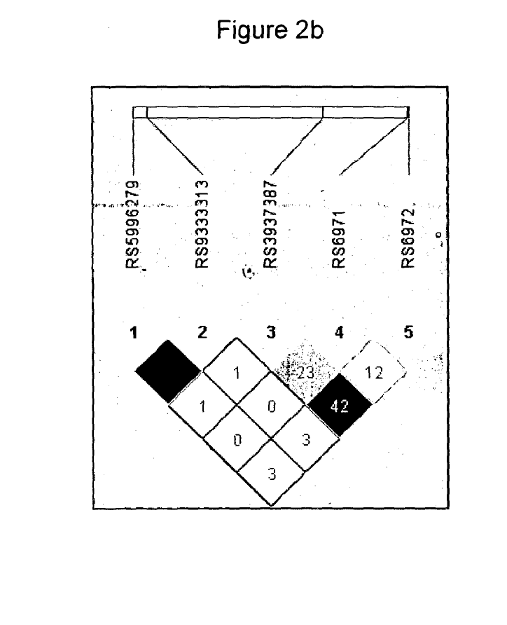

FIG. 2a. Single marker association test results for all 20 polymorphisms analysed using the additive genetic model and multiple-testing adjusted p-values. The x-axis shows the polymorphisms genotyped for each gene as obtained from the public domain (dbSNP: http://www.ncbi.nlm.nih.gov/projects/SNP/). For BZRAP1 and TSPO, SNPs are ordered on the x-axis (left to right) 5′ to 3′. All five genes are located on different chromosomes.

FIG. 2b. Pairwise LD plot for the 5 TSPO polymorphisms analysed and the inter-SNP r2 values in each box indicating the level of correlation between each pair of polymorphisms. In this picture from Haploview (18). LD values are multiplied by 100 i.e complete LD (r2=1) would be equal to 100.

FIG. 3: overview of TSPO radioligands for PET

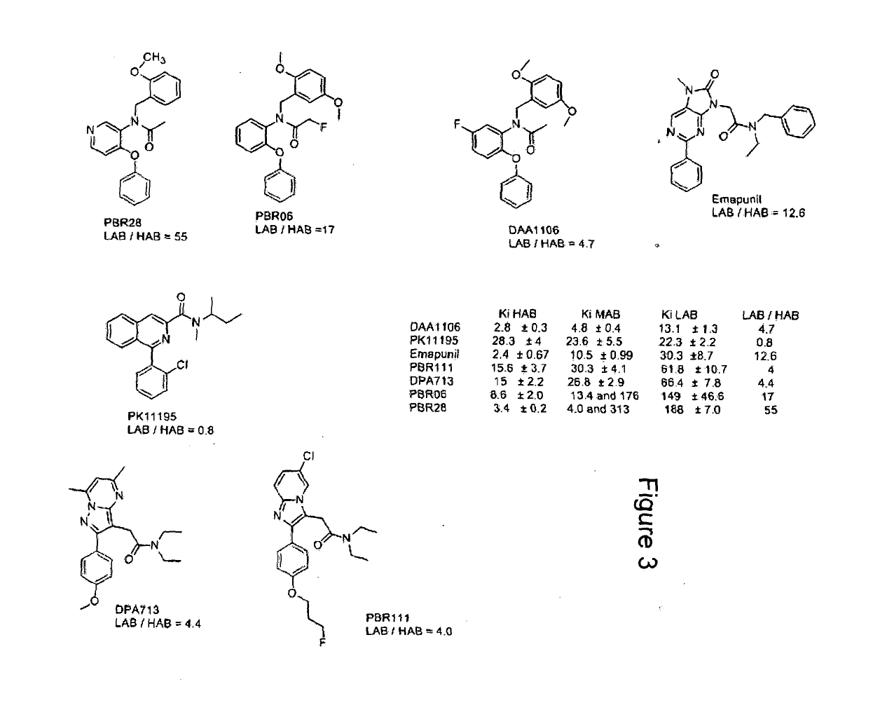

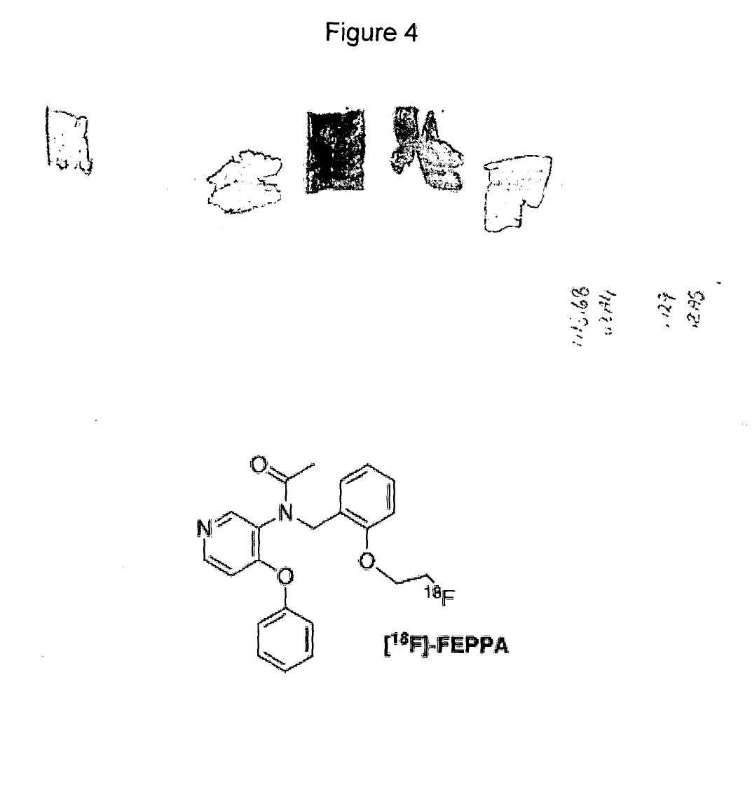

FIG. 4: Autoradiograhpy (above) with [¹⁸F]FEPPA (structure below). The sample slides in the figure have been previously characterised with [³H]PBR28 and are all from HABs and MABs, but for the slide second from the left which is a LAB. The lack of signal for the LAB with [¹⁸F]FEPPA in this experiment demonstrates that the binding of FEPPA for the TSPO differs between HABs, MABs and LABs.

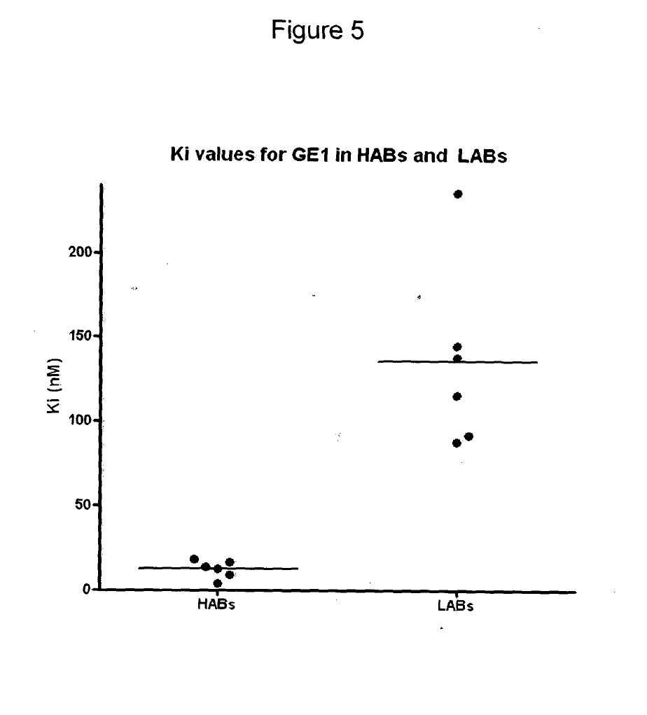

FIG. 5: Ki values for GE1 in HABs and LABs

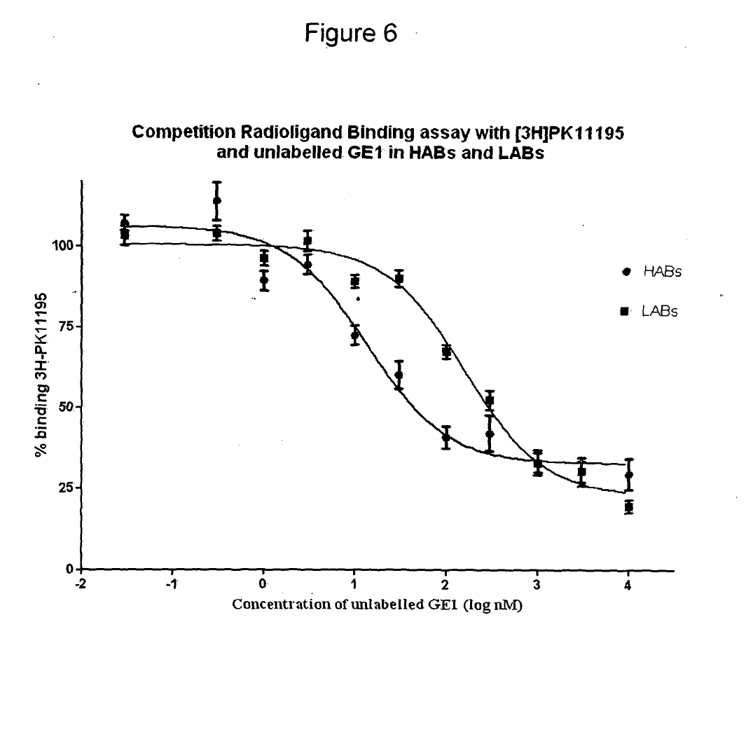

FIG. 6: Competition radioligand binding assay with [3H] PK11195 and unlabelled GE1 in HABs and LABs

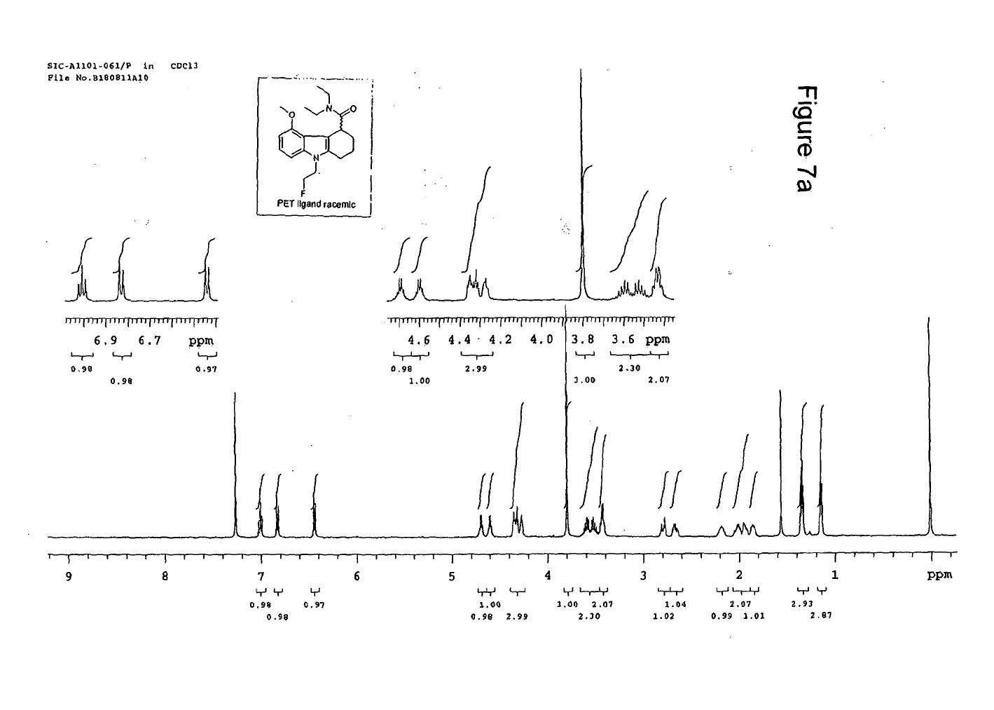

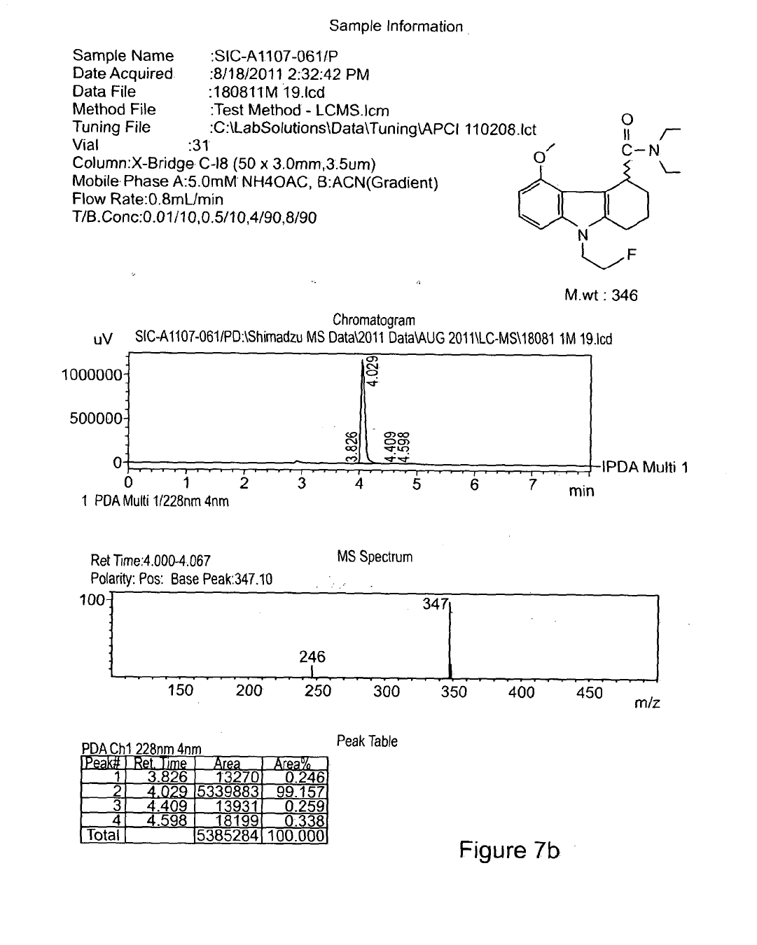

FIG. 7 a and b: characterisation of synthesised GE1 by LC-MS and NMR.

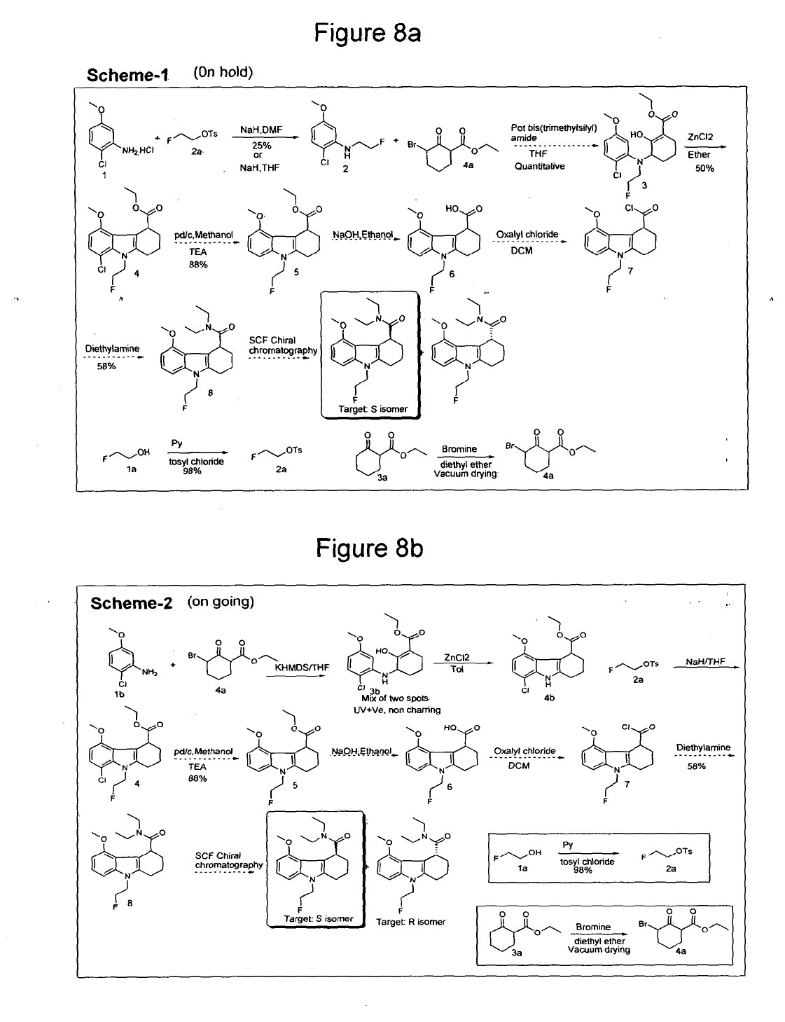

FIG. 8 a and b: synthesis of GE1: original (a) and modified (b) synthesis scheme

FIG. 9: Binding affinity of vinpocetine for TSPO in HABs and LABs in human brain (p<0.05). Competition assays with [³H]PK11195 and unlabelled Vinpocetine. Each data point represents the mean value for all 5 subjects within each group. Error bars represent standard error of the mean. Abbreviations: HABs; High affinity binders. LABs; Low affinity binders. MABs; Mixed affinity binders. See Example 4.

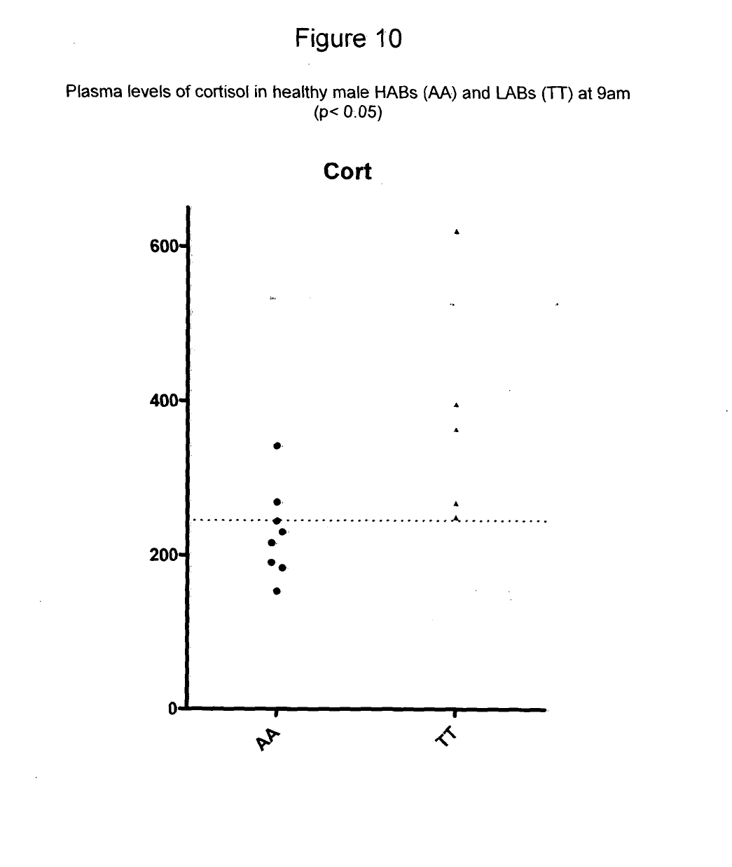

FIG. 10: Fold-increase in plasma cortisol following ACTH administration (p<0.05) indicating that TPSO genotype is reflected functionally in adrenal gland function. In this study using healthy male volunteers, venous blood samples were drawn every 15 minutes from approximately 9 am-11 am and cortisol was measured. ACTH (the gold standard stimulus test to induce cortisol release) was then administered and cortisol was subsequently measured every 15 minutes for a further 90 minutes. The increase in cortisol following ACTH administration was expressed as:

(area under the cortisol curve post ACTH administration)/(area under the cortisol curve pre ACTH administration).

Each point on the graph represents a different subject.

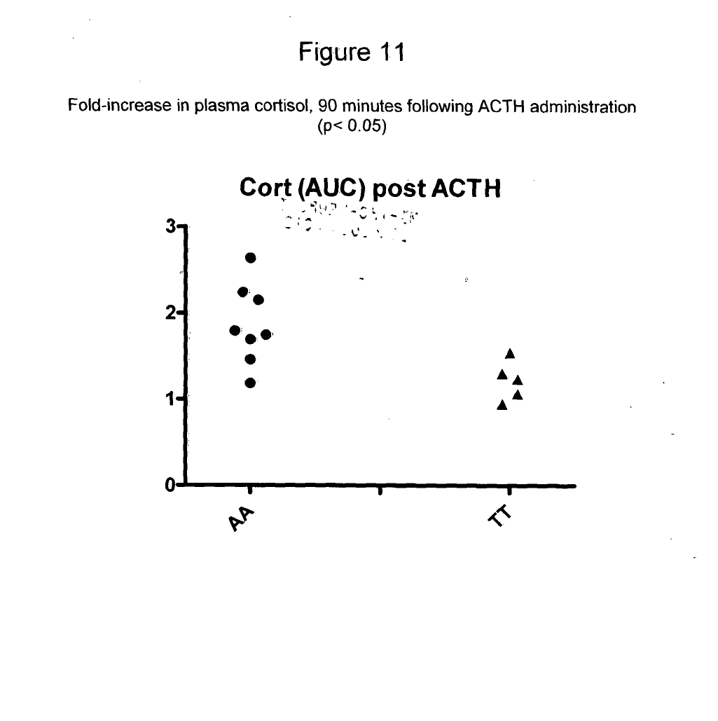

FIG. 11: Plasma levels of cortisol in healthy male HABs (AA) and LABs (TT) at 9 am (p<0.05) indicating that TPSO genotype is reflected functionally in adrenal gland function. In this study using healthy male volunteers, venous blood samples were drawn every 15 minutes from approximately 9 am-11 am and cortisol was measured. The graphs shows the mean plasma cortisol concentration for each subject.

### EXAMPLE 1

### An 18 kDa Translocator Protein (TSPO) Polymorphism Explains Differences in Relative Binding Affinity of the PET Radioligand PBR28

[¹¹C]PBR28 binds the 18 kDa Translocator Protein (TSPO) and is used in positron emission tomography (PET) to detect microglial activation. However, quantitative interpretations of signal are confounded by large inter-individual variability in binding affinity, which displays a trimodal distribution compatible with a co-dominant genetic trait.

Here, we tested directly for an underlying genetic mechanism to explain this. Methods Binding affinity of PBR28 was measured in platelets isolated from 41 human subjects and tested for association with polymorphisms in TSPO and genes encoding other proteins in the TSPO complex. Results Complete agreement was observed between the TSPO Ala147Thr genotype and PBR28 binding affinity phenotype (adjusted p-value=5.7×10⁻¹⁰ with low and medium affinity binders grouped together, or p-value=3.1×10⁻¹³ with low, medium and high affinity binders grouped separately).

### Conclusions

The TSPO Ala147Thr polymorphism predicts PBR28 binding affinity in human platelets. As all second generation TSPO PET radioligands tested hitherto display a trimodal distribution in binding affinity analogous to PBR28, testing for this polymorphism may allow quantitative interpretation of TSPO PET studies with these radioligands.

### Materials and Methods

The study protocol, volunteer information and informed consent forms were approved by the local Research Ethics Committee. Forty-one healthy volunteers (29 male, 12 female, mean age 36.3±1.4 years) were recruited. Ethnicity was self reported as 37/41 Caucasian, 2/41 Asian, 1/41 mixed Caucasian/Asian and 1/41 Hispanic. Venous blood (50 ml) was drawn into EDTA-containing tubes, and separated into a lymphocyte-rich bottom layer (for genetic analysis) and platelet-rich top layer by centrifugation (180×g, 15 min, 4° C.). The platelet-rich layer (for binding assays) was re-centrifuged (1800×g, 15 min, 4° C.) to produce a platelet-containing pellet. Platelet membranes were prepared as previously described (5). To measure binding affinity, aliquots of membrane suspension were incubated with 5 nM [³H]PK11195 (Perkin Elmer, UK) and one of 12 concentrations (0.1 nM-100 μM) of unlabelled PBR28 (Borochem, France) as previously described (5).

### DNA Extraction

Genomic DNA was extracted (by Gen-Probe, UK) from approximately 20 ml of lymphocyte-enriched blood product (see membrane preparation) using a chlorinated DNA extraction protocol and resuspended in 10 mM Tris/0.1 mM EDTA pH 8.0. Twenty nanograms of each genomic DNA sample was plated and lyophilised in a 96-well microtiter plate for each polymorphism tested. All samples were duplicated on each plate as a quality control measure.

### Polymorphism Selection and Genotyping

A total of 58 polymorphisms (both single nucleotide changes and insertions/deletions) with a perceived effect on protein function were selected from known polymorphisms in the TSPO gene and in genes encoding proteins directly associated with TSPO in the TSPO complex (VDAC1, VDAC2, VDAC3, ANT (SLC25A4), PRAX1 (BZRAP1), PAP7 (ACBD3)) (Table 2). The polymorphisms were genotyped using TaqMan® (Applied Biosystems, CA, USA)), Luminex-based Flow Assorted SNP Typing (FAST) (7), direct sequencing or polymerase chain reaction (PCR) fragment analysis (see Supplementary Methods for more details on assay conditions and quality control measures). Of the 58 genotyped polymorphisms, 38 were found to be monomorphic in the study sample, therefore only 20 polymorphisms were analysed for association with the ligand binding phenotype (see Table 2). Table 3 shows analysis using three groups: LABs, MABs and HABs

TABLE 2

| | | | | | | | | | Single Marker | |
|---|---|---|---|---|---|---|---|---|---|---|
| | | | | | | NCBI build | | | Association Test | |
| | | | Nucleotide | Amino | Chro- | 38 | Geno- | | Additive | Additive |
| Polymorphism | Gene | Gene | Base | Acid | mo- | Position | typing | Mono- | LAB vs | LAB + MAB |
| ID | Symbols | Region | Change | Change | some | (bp) | Technology | morphic? | MAB + HAB | vs HAB |
| rs3211100 | ACBD3 | Exon 8 | T > G | Glu490Asp | 1 | 224401051 | FAST | Yes | | |
| | | Coding | | | | | | | | |
| | | Sequence | | | | | | | | |
| rs17855715 | ACBD3 | Exon 8 | C > T | Lys473Arg | 1 | 224401103 | FAST | Yes | | |
| | | Coding | | | | | | | | |
| | | Sequence | | | | | | | | |
| rs1219666 | ACBD3 | Intron 7 | A > G | | 1 | 224401201 | FAST | Yes | | |
| | | Splicing | | | | | | | | |
| | | Lariat | | | | | | | | |
| rs9730340 | ACBD3 | Exon 4 | A > T | Glu193Asp | 1 | 224416004 | FAST | Yes | | |
| | | Coding | | | | | | | | |
| | | Sequence | | | | | | | | |
| rs2306120 | ACBD3 | Exon 3 | T > G | Glu187Asp | 1 | 224419121 | FAST | No | 0.421 | 1.000 |
| | | Coding | | | | | | | | |
| | | Sequence | | | | | | | | |
| rs2301868 | BZRAP1 | Exon 29 | G > A | Gly1770Glu | 17 | 53737476 | FAST | No | 1.000 | 1.000 |
| | | Coding | | | | | | | | |
| | | Sequence | | | | | | | | |
| rs9893754 | BZRAP1 | Exon 29 | G > T | Asn1764Thr | 17 | 53737494 | FAST | Yes | | |
| | | Coding | | | | | | | | |
| | | Sequence | | | | | | | | |
| rs3744103 | BZRAP1 | Intron | G > A | | 17 | 53738052 | FAST | No | 1.000 | 0.643 |
| | | 26 | | | | | | | | |
| | | Splicing | | | | | | | | |
| | | Lariat | | | | | | | | |
| rs34184983 | BZRAP1 | Intron | A > del | | 17 | 53738700 | FRET | Yes | | |
| | | 25 | | | | | | | | |
| | | Spice | | | | | | | | |
| | | Donor | | | | | | | | |
| rs2877876 | BZRAP1 | Intron | A > G | | 17 | 53739347 | FRET | Yes | | |
| | | 23 | | | | | | | | |
| | | Splicing | | | | | | | | |
| | | Lariat | | | | | | | | |
| rs11652816 | BZRAP1 | Intron | A > G | | 17 | 53739373 | FAST | No | 1.000 | 0.656 |
| | | 23 | | | | | | | | |
| | | Splicing | | | | | | | | |
| | | Lariat | | | | | | | | |
| rs72839988 | BZRAP1 | Intron | C > T | | 17 | 53740144 | FAST | No | 1.000 | 0.543 |
| | | 22 | | | | | | | | |
| | | Splicing | | | | | | | | |
| | | Lariat | | | | | | | | |
| rs34937759 | BZRAP1 | Intron | G > del | | 17 | 53740354 | FRET | Yes | | |
| | | 21 | | | | | | | | |
| | | Splicing | | | | | | | | |
| | | Lariat | | | | | | | | |
| rs61732758 | BZRAP1 | Exon 21 | C > T | Pro1468Ser | 17 | 53740996 | Sequencing | Yes | | |
| | | Coding | | | | | | | | |
| | | Sequence | | | | | | | | |
| rs61754146 | BZRAP1 | Exon 21 | A > G | Pro1299Leu | 17 | 53741556 | FAST | Yes | | |
| | | Coding | | | | | | | | |
| | | Sequence | | | | | | | | |
| rs61741210 | BZRAP1 | Exon 21 | C > T | Asp1241Asn | 17 | 53741731 | FAST | No | 1.000 | 0.539 |
| | | Coding | | | | | | | | |
| | | Sequence | | | | | | | | |
| rs3744101 | BZRAP1 | Exon 20 | C > T | Arg1193Cys | 17 | 53742461 | FAST | Yes | | |
| | | Coding | | | | | | | | |
| | | Sequence | | | | | | | | |
| rs34126526 | BZRAP1 | Intron | C > del | | 17 | 53743146 | FRET | Yes | | |
| | | 18 | | | | | | | | |
| | | Splicing | | | | | | | | |
| | | Lariat | | | | | | | | |
| rs2680704 | BZRAP1 | Exon 18 | G > C | Ala1080Pro | 17 | 53743237 | FAST | No | 1.000 | 0.656 |
| | | Coding | | | | | | | | |
| | | Sequence | | | | | | | | |
| rs3744099 | BZRAP1 | Exon 18 | A > T | His1058Leu | 17 | 53743302 | FAST | No | 1.000 | 1.000 |
| | | Coding | | | | | | | | |
| | | Sequence | | | | | | | | |
| rs9674726 | BZRAP1 | Intron | C > T | | 17 | 53743625 | FAST | Yes | | |
| | | 17 | | | | | | | | |
| | | Splicing | | | | | | | | |
| | | Lariat | | | | | | | | |
| rs2072144 | BZRAP1 | Intron | A > G | | 17 | 53743926 | FAST | No | 1.000 | 0.760 |
| | | 17 | | | | | | | | |
| | | Splice | | | | | | | | |
| | | Donor | | | | | | | | |
| rs9913145 | BZRAP1 | Exon 16 | C > T | Gln757Arg | 17 | 53744731 | FAST | No | 1.000 | 0.738 |
| | | Coding | | | | | | | | |
| | | Sequence | | | | | | | | |
| rs9783836 | BZRAP1 | Intron | A > G | | 17 | 53745132 | FAST | Yes | | |
| | | 15 | | | | | | | | |
| | | Splicing | | | | | | | | |
| | | Lariat | | | | | | | | |
| rs2072143 | BZRAP1 | Intron | A > G | | 17 | 53748547 | FAST | No | 1.000 | 1.000 |
| | | 14 | | | | | | | | |
| | | Splicing | | | | | | | | |
| | | Lariat | | | | | | | | |
| rs2072147 | BZRAP1 | Exon 13 | G > A | Ala526Thr | 17 | 53750756 | FAST | No | 1.000 | 1.000 |
| | | Coding | | | | | | | | |
| | | Sequence | | | | | | | | |
| rs5821222 | BZRAP1 | Exon 13 | G > del | Thr519— | 17 | 53750776 | FRET | Yes | | |
| | | Coding | | | | | | | | |
| | | Sequence | | | | | | | | |
| rs11652447 | BZRAP1 | Exon 12 | C > T | Ser456Asn | 17 | 53751602 | FAST | Yes | | |
| | | Coding | | | | | | | | |
| | | Sequence | | | | | | | | |
| rs2072145 | BZRAP1 | Exon 12 | A > G | Gln454Arg | 17 | 53751608 | FAST | No | 1.000 | 1.000 |
| | | Coding | | | | | | | | |
| | | Sequence | | | | | | | | |
| rs61747227 | BZRAP1 | Exon 10 | C > G | Gln420His | 17 | 53752924 | FAST | Yes | | |
| | | Coding | | | | | | | | |
| | | Sequence | | | | | | | | |
| rs9894786 | BZRAP1 | Intron 6 | C > T | | 17 | 53755452 | FAST | Yes | | |
| | | Splicing | | | | | | | | |
| | | Lariat | | | | | | | | |
| rs1042560 | SLC25A4 | Exon 2 | T > G | Val58Leu | 4 | 186302972 | FAST | Yes | | |
| | | Coding | | | | | | | | |
| | | Sequence | | | | | | | | |
| rs28999114 | SLC25A4 | Exon 2 | A > G | Asp104Gly | 4 | 186303111 | FAST | Yes | | |
| | | Coding | | | | | | | | |
| | | Sequence | | | | | | | | |
| rs62619215 | SLC25A4 | Exon 2 | AG > | Lys147— | 4 | 186303241 | FRET | Yes | | |
| | | Coding | del | | | | | | | |
| | | Sequence | | | | | | | | |
| rs62620230 | SLC25A4 | Exon 2 | —> C | Ala149— | 4 | 186303246 | Sequencing | Yes | | |
| | | Coding | | | | | | | | |
| | | Sequence | | | | | | | | |
| rs55820580 | SLC25A4 | Exon 3 | C > G | Arg236Gly | 4 | 186304014 | FAST | Yes | | |
| | | Coding | | | | | | | | |
| | | Seguence | | | | | | | | |
| rs113193775 | SLC25A4 | Intron 3 | C > G | | 4 | 186304048 | FAST | Yes | | |
| | | Splice | | | | | | | | |
| | | Donor | | | | | | | | |
| rs111811353 | SLC25A4 | Intron 3 | A > G | | 4 | 186304049 | FAST | Yes | | |
| | | Splice | | | | | | | | |
| | | Donor | | | | | | | | |
| rs5996279 | TSPO | Promoter | A > C | | 22 | 41876677 | FAST | No | 1.000 | 1.000 |
| rs9333313 | TSPO | Promoter | TCGC > | | 22 | 41877316 | FRET | No | 1.000 | 1.000 |
| | | | del | | | | | | | |
| rs3937387 | TSPO | Intron 1 | C > G | | 22 | 41885138 | FAST | No | 0.115 | 0.055 |
| | | Splicing | | | | | | | | |
| | | Lariat | | | | | | | | |
| rs34936584 | TSPO | Intron 2 | T < del | | 22 | 41886995 | FRET | Yes | | |
| | | Splicing | | | | | | | | |
| | | Lariat | | | | | | | | |
| rs71758389 | TSPO | Exon 3 | 1612bp | Trp107— | 22 | 41887140 | PCR & | Yes | | |
| | | Coding | deletion | | | | agarose | | | |
| | | Sequence | | | | | gel | | | |
| | | Splice | | | | | electrophoresis | | | |
| | | Donor | | | | | | | | |
| rs9333341 | TSPO | Exon 3 | C > G | Leu44Val | 22 | 41888842 | FAST | Yes | | |
| | | Coding | | | | | | | | |
| | | Sequence | | | | | | | | |
| rs6971 | TSPO | Exon 4 | C > T^{a} | Ala147Thr | 22 | 41888870 | FRET | No | 0.001 | 2.838E−11 |
| | | Coding | | | | | | | | |
| | | Sequence | | | | | | | | |
| rs6972 | TSPO | Exon 4 | G > A | Arg162His | 22 | 41888916 | FRET | No | 0.606 | 0.038 |
| | | Coding | | | | | | | | |
| | | Sequence | | | | | | | | |
| rs8192467 | TSPO | Exon 4 | G > T | Arg166Leu | 22 | 41888928 | FRET | Yes | | |
| | | Coding | | | | | | | | |
| | | Sequence | | | | | | | | |
| rs9333342 | TSPO | Exon 4 | C > G | Glu169Gln | 22 | 41888936 | FRET | Yes | | |
| | | Coding | | | | | | | | |
| | | Sequence | | | | | | | | |
| | | Splice | | | | | | | | |
| | | Donor | | | | | | | | |
| rs6892975 | VDAC1 | Intron 1 | G > T | | 5 | 133356632 | FAST | Yes | | |
| | | Splicing | | | | | | | | |
| | | Lariat | | | | | | | | |
| rs11537680 | VDAC2 | Exon 5 | C > T | Thr66Ile | 10 | 76648873 | FAST | Yes | | |
| | | Coding | | | | | | | | |
| | | Sequence | | | | | | | | |
| rs60564032 | VDAC2 | Exon 7 | G > del | Asp188— | 10 | 76650712 | Sequencing | Yes | | |
| | | Coding | | | | | | | | |
| | | Sequence | | | | | | | | |
| rs34070926 | VDAC2 | Exon 10 | C > G | Gly255Ala | 10 | 76659436 | FAST | Yes | | |
| | | Coding | | | | | | | | |
| | | Sequence | | | | | | | | |
| rs74146301 | VDAC2 | Intron | A > G | | 10 | 76660652 | FAST | No | 1.000 | 1.000 |
| | | 10 | | | | | | | | |
| | | Splicing | | | | | | | | |
| | | Lariat | | | | | | | | |
| rs62508038 | VDAC3 | Exon 6 | C > G | Thr51Arg | 8 | 42375421 | FAST | Yes | | |
| | | Coding | | | | | | | | |
| | | Sequence | | | | | | | | |
| rs34738318 | VDAC3 | Intron 6 | C > del | | 8 | 42376301 | FRET | Yes | | |
| | | Splicing | | | | | | | | |
| | | Lariat | | | | | | | | |
| rs35475165 | VDAC3 | Intron 6 | A > del | | 8 | 42376311 | Sequencing | Yes | | |
| | | Splicing | | | | | | | | |
| | | Lariat | | | | | | | | |
| rs3736549 | VDAC3 | Intron 7 | A > G | | 8 | 42378456 | FAST | No | 1.000 | 1.000 |
| | | Splicing | | | | | | | | |
| | | Lariat | | | | | | | | |
| rs11558228 | VDAC3 | Exon 8 | G > T | Gly148Trp | 8 | 42378581 | FAST | Yes | | |
| | | Coding | | | | | | | | |
| | | Sequence | | | | | | | | |

| | Single Marker | | | | | | | | Yoruba |
|---|---|---|---|---|---|---|---|---|---|
| | Association Test | | | | | | | JP | (AF) |
| | | Dominant | | | Study | Caucasian | CN | Allele | Allele |
| | Dominant | LAB + MAB | HWE | | Allele | Allele | Allele | Freq | Freq |
| Polymorphism | LAB vs | vs | p- | | Freq (# | Freq (# | Freq (# | (# | (# |
| ID | MAB + HAB | HAB | value | Alleles | chr) | chr) | chr) | chr) | chr) |
| rs3211100 | | | | G | 1.000 | 1.000 | 1.000 | 1.000 | 1.000 |
| | | | | | (82) | (116) | (86) | (88) | (116) |
| | | | | T | 0.000 | 0.000 | 0.000 | 0.000 | 0.000 |
| | | | | | (0) | (0) | (0) | (0) | (0) |
| rs17855715 | | | | T | 1.000 | 1.000 | 1.000 | 1.000 | 1.000 |
| | | | | | (82) | (116) | (88) | (88) | (118) |
| | | | | C | 0.000 | 0.000 | 0.000 | 0.000 | 0.000 |
| | | | | | (0) | (0) | (0) | (0) | (0) |
| rs1219666 | | | | A | 1.000 | 1.000 | 1.000 | 1.000 | 1.000 |
| | | | | | (82) | (116) | (88) | (88) | (118) |
| | | | | G | 0.000 | 0.000 | 0.000 | 0.000 | 0.000 |
| | | | | | (0) | (0) | (0) | (0) | (0) |
| rs9730340 | | | | T | 1.000 | 1.000 | 1.000 | 1.000 | 1.000 |
| | | | | | (82) | (116) | (84) | (84) | (118) |
| | | | | A | 0.000 | 0.000 | 0.000 | 0.000 | 0.000 |
| | | | | | (0) | (0) | (0) | (0) | (0) |
| rs2306120 | 0.146 | 1.000 | 1.000 | T | 0.378 | 0.371 | 0.477 | 0.580 | 0.345 |
| | | | | | (31) | (43) | (41) | (51) | (40) |
| | | | | G | 0.622 | 0.529 | 0.523 | 0.420 | 0.655 |
| | | | | | (51) | (73) | (45) | (37) | (76) |
| rs2301868 | 1.000 | 1.000 | 1.000 | A | 0.024 | 0.052 | 0.159 | 0.068 | 0.000 |
| | | | | | (2) | (6) | (14) | (6) | (0) |
| | | | | G | 0.976 | 0.948 | 0.841 | 0.932 | 1.000 |
| | | | | | (80) | (110) | (74) | (82) | (118) |
| rs9893754 | | | | T | 1.000 | 1.000 | 1.000 | 1.000 | 1.000 |
| | | | | | (82) | (116) | (86) | (88) | (116) |
| | | | | G | 0.000 | 0.000 | 0.000 | 0.000 | 0.000 |
| | | | | | (0) | (0) | (0) | (0) | (0) |
| rs3744103 | 1.000 | 0.643 | 1.000 | A | 0.075 | 0.121 | 0.159 | 0.068 | 0.076 |
| | | | | | (6) | (14) | (14) | (6) | (9) |
| | | | | G | 0.925 | 0.879 | 0.841 | 0.932 | 0.924 |
| | | | | | (74) | (102) | (74) | (82) | (109) |
| rs34184983 | | | | del | 1.000 | 1.000 | 1.000 | 1.000 | 1.000 |
| | | | | | (82) | (116) | (90) | (88) | (120) |
| | | | | A | 0.000 | 0.000 | 0.000 | 0.000 | 0.000 |
| | | | | | (0) | (0) | (0) | (0) | (0) |
| rs2877876 | | | | G | 1.000 | 1.000 | 1.000 | 1.000 | 1.000 |
| | | | | | (82) | (116) | (90) | (86) | (120) |
| | | | | A | 0.000 | 0.000 | 0.000 | 0.000 | 0.000 |
| | | | | | (0) | (0) | (0) | (0) | (0) |
| rs11652816 | 1.000 | 0.520 | 0.647 | A | 0.756 | 0.629 | 0.774 | 0.744 | 0.712 |
| | | | | | (62) | (73) | (65) | (64) | (84) |
| | | | | G | 0.244 | 0.371 | 0.226 | 0.256 | 0.288 |
| | | | | | (20) | (43) | (19) | (22) | (34) |
| rs72839988 | 1.000 | 0.543 | 1.000 | C | 0.925 | 0.879 | 0.852 | 0.909 | 0.873 |
| | | | | | (74) | (102) | (75) | (80) | (103) |
| | | | | T | 0.075 | 0.121 | 0.148 | 0.091 | 0.127 |
| | | | | | (6) | (14) | (13) | (8) | (15) |
| rs34937759 | | | | del | 1.000 | 1.000 | 1.000 | 1.000 | 1.000 |
| | | | | | (82) | (116) | (90) | (88) | (120) |
| | | | | G | 0.000 | 0.000 | 0.000 | 0.000 | 0.000 |
| | | | | | (0) | (0) | (0) | (0) | (0) |
| rs61732758 | | | | C | 1.000 | nd | nd | nd | nd |
| | | | | | (82) | | | | |
| | | | | T | 0.000 | | | | |
| | | | | | (0) | | | | |
| rs61754146 | | | | G | 1.000 | 1.000 | 1.000 | 1.000 | 1.000 |
| | | | | | (82) | (116) | (88) | (88) | (118) |
| | | | | A | 0.000 | 0.000 | 0.000 | 0.000 | 0.000 |
| | | | | | (0) | (0) | (0) | (0) | (0) |
| rs61741210 | 1.000 | 0.539 | 1.000 | C | 0.963 | 0.931 | 1.000 | 1.000 | 1.000 |
| | | | | | (79) | (108) | (88) | (88) | (118) |
| | | | | T | 0.037 | 0.069 | 0.000 | 0.000 | 0.000 |
| | | | | | (3) | (8) | (0) | (0) | (0) |
| rs3744101 | | | | C | 1.000 | 1.000 | 1.000 | 1.000 | 1.000 |
| | | | | | (82) | (116) | (88) | (88) | (118) |
| | | | | T | 0.000 | 0.000 | 0.000 | 0.000 | 0.000 |
| | | | | | (0) | (0) | (0) | (0) | (0) |
| rs34126526 | | | | del | 1.000 | 1.000 | 1.000 | 1.000 | 1.000 |
| | | | | | (82) | (116) | (90) | (88) | (120) |
| | | | | C | 0.000 | 0.000 | 0.000 | 0.000 | 0.000 |
| | | | | | (0) | (0) | (0) | (0) | (0) |
| rs2680704 | 1.000 | 0.520 | 0.647 | C | 0.756 | 0.629 | 0.789 | 0.779 | 0.771 |
| | | | | | (62) | (73) | (71) | (67) | (91) |
| | | | | G | 0.244 | 0.371 | 0.211 | 0.221 | 0.229 |
| | | | | | (20) | (43) | (19) | (19) | (27) |
| rs3744099 | 1.000 | 1.000 | 1.000 | A | 0.951 | 0.948 | 0.940 | 0.833 | 1.000 |
| | | | | | (78) | (110) | (79) | (70) | (118) |
| | | | | T | 0.049 | 0.052 | 0.060 | 0.167 | 0.000 |
| | | | | | (4) | (6) | (5) | (14) | (0) |
| rs9674726 | | | | C | 1.000 | 1.000 | 1.000 | 1.000 | 0.741 |
| | | | | | (82) | (116) | (88) | (88) | (86) |
| | | | | T | 0.000 | 0.000 | 0.000 | 0.000 | 0.259 |
| | | | | | (0) | (0) | (0) | (0) | (30) |
| rs2072144 | 1.000 | 0.734 | 0.645 | A | 0.744 | 0.638 | 0.761 | 0.773 | 0.681 |
| | | | | | (58) | (74) | (67) | (68) | (79) |
| | | | | G | 0.256 | 0.362 | 0.239 | 0.227 | 0.319 |
| | | | | | (20) | (42) | (21) | (20) | (37) |
| rs9913145 | 1.000 | 1.000 | 1.000 | C | 0.073 | 0.112 | 0.261 | 0.221 | 0.250 |
| | | | | | (6) | (13) | (23) | (19) | (29) |
| | | | | T | 0.927 | 0.888 | 0.739 | 0.779 | 0.750 |
| | | | | | (76) | (103) | (65) | (67) | (87) |
| rs9783836 | | | | A | 0.000 | 0.000 | 0.000 | 0.000 | 0.093 |
| | | | | | (0) | (0) | (0) | (0) | (11) |
| | | | | G | 1.000 | 1.000 | 1.000 | 1.000 | 0.907 |
| | | | | | (82) | (116) | (88) | (86) | (107) |
| rs2072143 | 1.000 | 1.000 | 1.000 | A | 0.939 | 0.940 | 0.852 | 0.750 | 1.000 |
| | | | | | (77) | (109) | (75) | (66) | (118) |
| | | | | G | 0.061 | 0.060 | 0.148 | 0.250 | 0.000 |
| | | | | | (5) | (7) | (13) | (22) | (0) |
| rs2072147 | 1.000 | 1.000 | 1.000 | A | 0.024 | 0.050 | 0.216 | 0.125 | 0.017 |
| | | | | | (2) | (7) | (19) | (11) | (2) |
| | | | | G | 0.976 | 0.940 | 0.784 | 0.875 | 0.983 |
| | | | | | (80) | (109) | (69) | (77) | (116) |
| rs5821222 | | | | G | 1.000 | 1.000 | 1.000 | 1.000 | 1.000 |
| | | | | | (82) | (116) | (90) | (88) | (120) |
| | | | | del | 0.000 | 0.000 | 0.000 | 0.000 | 0.000 |
| | | | | | (0) | (0) | (0) | (0) | (0) |
| rs11652447 | | | | C | 1.000 | 1.000 | 1.000 | 1.000 | 1.000 |
| | | | | | (82) | (116) | (84) | (84) | (118) |
| | | | | T | 0.000 | 0.000 | 0.000 | 0.000 | 0.000 |
| | | | | | (0) | (0) | (0) | (0) | (0) |
| rs2072145 | 1.000 | 1.000 | 1.000 | A | 0.976 | 0.940 | 0.784 | 0.875 | 1.000 |
| | | | | | (80) | (109) | (69) | (77) | (118) |
| | | | | G | 0.024 | 0.060 | 0.216 | 0.125 | 0.000 |
| | | | | | (2) | (7) | (19) | (11) | (0) |
| rs61747227 | | | | C | 1.000 | 1.000 | 1.000 | 1.000 | 0.966 |
| | | | | | (82) | (116) | (90) | (86) | (114) |
| | | | | G | 0.000 | 0.000 | 0.000 | 0.000 | 0.034 |
| | | | | | (0) | (0) | (0) | (0) | (4) |
| rs9894786 | | | | C | 1.000 | 1.000 | 1.000 | 1.000 | 0.907 |
| | | | | | (82) | (116) | (88) | (88) | (107) |
| | | | | T | 0.000 | 0.000 | 0.000 | 0.000 | 0.093 |
| | | | | | (0) | (0) | (0) | (0) | (11) |
| rs1042560 | | | | G | 1.000 | 1.000 | 1.000 | 1.000 | 1.000 |
| | | | | | (82) | (116) | (86) | (88) | (116) |
| | | | | T | 0.000 | 0.000 | 0.000 | 0.000 | 0.000 |
| | | | | | (0) | (0) | (0) | (0) | (0) |
| rs28999114 | | | | A | 1.000 | 1.000 | 1.000 | 1.000 | 1.000 |
| | | | | | (82) | (116) | (88) | (88) | (118) |
| | | | | G | 0.000 | 0.000 | 0.000 | 0.000 | 0.000 |
| | | | | | (0) | (0) | (0) | (0) | (0) |
| rs62619215 | | | | AG | 1.000 | 1.000 | 1.000 | 1.000 | 1.000 |
| | | | | | (82) | (116) | (90) | (88) | (120) |
| | | | | del | 0.000 | 0.000 | 0.000 | 0.000 | 0.000 |
| | | | | | (0) | (0) | (0) | (0) | (0) |
| rs62620230 | | | | C | 1.000 | nd | nd | nd | nd |
| | | | | | (82) | | | | |
| | | | | del | 0.000 | | | | |
| | | | | | (0) | | | | |
| rs55820580 | | | | C | 1.000 | 1.000 | 1.000 | 1.000 | 1.000 |
| | | | | | (82) | (116) | (90) | (86) | (118) |
| | | | | G | 0.000 | 0.000 | 0.000 | 0.000 | 0.000 |
| | | | | | (0) | (0) | (0) | (0) | (0) |
| rs113193775 | | | | C | 1.000 | 1.000 | 1.000 | 1.000 | 1.000 |
| | | | | | (82) | (16) | (90) | (86) | (118) |
| | | | | G | 0.000 | 0.000 | 0.000 | 0.000 | 0.000 |
| | | | | | (0) | (0) | (0) | (0) | (0) |
| rs111811353 | | | | A | 1.000 | 1.000 | 1.000 | 1.000 | 1.000 |
| | | | | | (82) | (116) | (88) | (88) | (118) |
| | | | | G | 0.000 | 0.000 | 0.000 | 0.000 | 0.000 |
| | | | | | (0) | (0) | (0) | (0) | (0) |
| rs5996279 | 1.000 | 1.000 | 1.000 | A | 0.988 | 1.000 | 1.000 | 1.000 | 0.612 |
| | | | | | (81) | (116) | (86) | (88) | (71) |
| | | | | C | 0.012 | 0.000 | 0.000 | 0.000 | 0.388 |
| | | | | | (1) | (0) | (0) | (0) | (45) |
| rs9333313 | 1.000 | 1.000 | 1.000 | del | 0.012 | 0.000 | 0.000 | 0.000 | 0.383 |
| | | | | | (1) | (0) | (0) | (0) | (46) |
| | | | | TCGC | 0.988 | 1.000 | 1.000 | 0.000 | 0.617 |
| | | | | | (81) | (116) | (90) | (88) | (74) |
| rs3937387 | 0.084 | 0.278 | 0.000 | C | 0.451 | 0.388 | 0.022 | 0.000 | 0.551 |
| | | | | | (37) | (45) | (2) | (0) | (65) |
| | | | | G | 0.549 | 0.612 | 0.978 | 1.000 | 0.449 |
| | | | | | (45) | (71) | (88) | (86) | (53) |
| rs34936584 | | | | del | 1.000 | 1.000 | 1.000 | 1.000 | 1.000 |
| | | | | | (82) | (116) | (90) | (88) | (120) |
| | | | | T | 0.000 | 0.000 | 0.000 | 0.000 | 0.000 |
| | | | | | (0) | (0) | (0) | (0) | (0) |
| rs71758389 | | | | 1612 bp | 1.000 | nd | nd | nd | nd |
| | | | | | (82) | | | | |
| | | | | — | 0.000 | | | | |
| | | | | | (0) | | | | |
| rs9333341 | | | | C | 1.000 | 1.000 | 1.000 | 1.000 | 1.000 |
| | | | | | (82) | (116) | (90) | (86) | (118) |
| | | | | G | 0.000 | 0.000 | 0.000 | 0.000 | 0.000 |
| | | | | | (0) | (0) | (0) | (0) | (0) |
| rs6971 | 0.111 | 2.838E−11 | 0.623 | C | 0.805 | 0.672 | 0.989 | 0.988 | 0.883 |
| | | | | | (66) | (78) | (89) | (85) | (106) |
| | | | | T | 0.195 | 0.328 | 0.011 | 0.012 | 0.117 |
| | | | | | (16) | (38) | (1) | (1) | (14) |
| rs6972 | 0.209 | 0.026 | 1.000 | A | 0.329 | 0.276 | 0.278 | 0.193 | 0.058 |
| | | | | | (27) | (32) | (25) | (17) | (7) |
| | | | | G | 0.671 | 0.724 | 0.722 | 0.807 | 0.942 |
| | | | | | (55) | (84) | (65) | (71) | (113) |
| rs8192467 | | | | G | 1.000 | 1.000 | 1.000 | 1.000 | 1.000 |
| | | | | | (82) | (116) | (90) | (86) | (120) |
| | | | | T | 0.000 | 0.000 | 0.000 | 0.000 | 0.000 |
| | | | | | (0) | (0) | (0) | (0) | (0) |
| rs9333342 | | | | G | 1.000 | 1.000 | 1.000 | 1.000 | 1.000 |
| | | | | | (82) | (116) | (90) | (88) | (120) |
| | | | | C | 0.000 | 0.000 | 0.000 | 0.000 | 0.000 |
| | | | | | (0) | (0) | (0) | (0) | (0) |
| rs6892975 | | | | T | 1.000 | 1.000 | 1.000 | 1.000 | 1.000 |
| | | | | | (82) | (116) | (86) | (88) | (116) |
| | | | | G | 0.000 | 0.000 | 0.000 | 0.000 | 0.000 |
| | | | | | (0) | (0) | (0) | (0) | (0) |
| rs11537680 | | | | C | 1.000 | 1.000 | 1.000 | 1.000 | 1.000 |
| | | | | | (82) | (116) | (88) | (88) | (118) |
| | | | | T | 0.000 | 0.000 | 0.000 | 0.000 | 0.000 |
| | | | | | (0) | (0) | (0) | (0) | (0) |
| rs60564032 | | | | G | 1.000 | nd | nd | nd | nd |
| | | | | | (82) | | | | |
| | | | | del | 0.000 | | | | |
| | | | | | (0) | | | | |
| rs34070926 | | | | G | 1.000 | 1.000 | 1.000 | 1.000 | 1.000 |
| | | | | | (82) | (116) | (90) | (86) | (118) |
| | | | | C | 0.000 | 0.000 | 0.000 | 0.000 | 0.000 |
| | | | | | (0) | (0) | (0) | (0) | (0) |
| rs74146301 | 1.000 | 1.000 | 0.084 | A | 0.061 | 0.060 | 0.045 | 0.057 | 0.203 |
| | | | | | (5) | (7) | (4) | (5) | (24) |
| | | | | G | 0.939 | 0.940 | 0.955 | 0.943 | 0.797 |
| | | | | | (77) | (109) | (84) | (83) | (94) |
| rs62508038 | | | | C | 1.000 | 1.000 | 1.000 | 1.000 | 1.000 |
| | | | | | (82) | (116) | (90) | (86) | (118) |
| | | | | G | 0.000 | 0.000 | 0.000 | 0.000 | 0.000 |
| | | | | | (0) | (0) | (0) | (0) | (0) |
| rs34738318 | | | | del | 1.000 | 1.000 | 1.000 | 1.000 | 1.000 |
| | | | | | (82) | (116) | (90) | (88) | (120) |
| | | | | C | 0.000 | 0.000 | 0.000 | 0.000 | 0.000 |
| | | | | | (0) | (0) | (0) | (0) | (0) |
| rs35475165 | | | | A | 1.000 | nd | nd | nd | nd |
| | | | | | (82) | | | | |
| | | | | — | 0.000 | | | | |
| | | | | | (0) | | | | |
| rs3736549 | 1.000 | 1.000 | 1.000 | A | 0.927 | 0.940 | 0.909 | 0.841 | 0.373 |
| | | | | | (76) | (109) | (80) | (74) | (44) |
| | | | | G | 0.073 | 0.060 | 0.091 | 0.159 | 0.627 |
| | | | | | (6) | (7) | (8) | (14) | (74) |
| rs11558228 | | | | G | 1.000 | 1.000 | 1.000 | 1.000 | 1.000 |
| | | | | | (82) | (116) | (86) | (88) | (116) |
| | | | | T | 0.000 | 0.000 | 0.000 | 0.000 | 0.000 |
| | | | | | (0) | (0) | (0) | (0) | (0) |

^{a}rs6971 SNP genotyping data is reflective of the antisense strand for both alleles. The alteration to the coding sequence is actually GCG [Ala] to ACG [Thr] as described in dbSNP (http://www.ncbi.nlm.nih.gov/projects/SNP/)

TABLE 3

| Polymorphism | Gene | | Nucleotide | Amino Acid | |
|---|---|---|---|---|---|
| ID | Symbols | Gene Region | Base Change | Change | Chromosome |
| rs3211100 | ACBD3 | Exon 8 Coding | T > G | Glu490Asp | 1 |
| | | Sequence | | | |
| rs17855715 | ACBD3 | Exon 8 Coding | C > T | Lys473Arg | 1 |
| | | Sequence | | | |
| rs1219666 | ACBD3 | Intron 7 Splicing Lariat | A > G | | 1 |
| rs9730340 | ACBD3 | Exon 4 Coding | A > T | Glu193Asp | 1 |
| | | Sequence | | | |
| rs2306120 | ACBD3 | Exon 3 Coding | T > G | Glu187Asp | 1 |
| | | Sequence | | | |
| rs2301868 | BZRAP1 | Exon 29 Coding | G > A | Gly1770Glu | 17 |
| | | Sequence | | | |
| rs9893754 | BZRAP1 | Exon 29 Coding | G > T | Asn1764Thr | 17 |
| | | Sequence | | | |
| rs3744103 | BZRAP1 | Intron 26 Splicing | G > A | | 17 |
| | | Lariat | | | |
| rs34184983 | BZRAP1 | Intron 25 Splice Donor | A > del | | 17 |
| rs2877876 | BZRAP1 | Intron 23 Splicing | A > G | | 17 |
| | | Lariat | | | |
| rs11652816 | BZRAP1 | Intron 23 Splicing | A > G | | 17 |
| | | Lariat | | | |
| rs72839988 | BZRAP1 | Intron 22 Splicing | C > T | | 17 |
| | | Lariat | | | |
| rs34937759 | BZRAP1 | Intron 21 Splicing | G > del | | 17 |
| | | Lariat | | | |
| rs61732758 | BZRAP1 | Exon 21 Coding | C > T | Pro1468Ser | 17 |
| | | Sequence | | | |
| rs61754146 | BZRAP1 | Exon 21 Coding | A > G | Pro1299Leu | 17 |
| | | Sequence | | | |
| rs61741210 | BZRAP1 | Exon 21 Coding | C > T | Asp1241Asn | 17 |
| | | Sequence | | | |
| rs3744101 | BZRAP1 | Exon 20 Coding | C > T | Arg1193Cys | 17 |
| | | Sequence | | | |
| rs34126526 | BZRAP1 | Intron 18 Splicing | C > del | | 17 |
| | | Lariat | | | |
| rs2680704 | BZRAP1 | Exon 18 Coding | G > C | Ala1080Pro | 17 |
| | | Sequence | | | |
| rs3744099 | BZRAP1 | Exon 18 Coding | A > T | His1058Leu | 17 |
| | | Sequence | | | |
| rs9674726 | BZRAP1 | Intron 17 Splicing | C > T | | 17 |
| | | Lariat | | | |
| rs2072144 | BZRAP1 | Intron 17 Splice Donor | A > G | | 17 |
| rs9913145 | BZRAP1 | Exon 16 Coding | C > T | Gln757Arg | 17 |
| | | Sequence | | | |
| rs9783836 | BZRAP1 | Intron 15 Splicing | A > G | | 17 |
| | | Lariat | | | |
| rs2072143 | BZRAP1 | Intron 14 Splicing | A > G | | 17 |
| | | Lariat | | | |
| rs2072147 | BZRAP1 | Exon 13 Coding | G > A | Ala526Thr | 17 |
| | | Sequence | | | |
| rs5821222 | BZRAP1 | Exon 13 Coding | G > del | Thr519— | 17 |
| | | Sequence | | | |
| rs11652447 | BZRAP1 | Exon 12 Coding | C > T | Ser456Asn | 17 |
| | | Sequence | | | |
| rs2072145 | BZRAP1 | Exon 12 Coding | A > G | Gln454Arg | 17 |
| | | Sequence | | | |
| rs61747227 | BZRAP1 | Exon 10 Coding | C > G | Gln420His | 17 |
| | | Sequence | | | |
| rs9894786 | BZRAP1 | Intron 6 Splicing Lariat | C > T | | 17 |
| rs1042560 | SLC25A4 | Exon 2 Coding | T > G | Val58Leu | 4 |
| | | Sequence | | | |
| rs28999114 | SLC25A4 | Exon 2 Coding | A > G | Asp104Gly | 4 |
| | | Sequence | | | |
| rs62619215 | SLC25A4 | Exon 2 Coding | AG > del | Lys147— | 4 |
| | | Sequence | | | |
| rs62620230 | SLC25A4 | Exon 2 Coding | —> C | Ala149— | 4 |
| | | Sequence | | | |
| rs55820580 | SLC25A4 | Exon 3 Coding | C > G | Arg236Gly | 4 |
| | | Sequence | | | |
| rs113193775 | SLC25A4 | Intron 3 Splice Donor | C > G | | 4 |
| rs111811353 | SLC25A4 | Intron 3 Splice Donor | A > G | | 4 |
| rs5996279 | TSPO | Promoter | A > C | | 22 |
| rs9333313 | TSPO | Promoter | TCGC > del | | 22 |
| rs3937387 | TSPO | Intron 1 Splicing Lariat | C > G | | 22 |
| rs34936584 | TSPO | Intron 2 Splicing Lariat | T > del | | 22 |
| rs71758389 | TSPO | Exon 3 Coding | 1612 bp | Trp107— | 22 |
| | | Sequence Splice | deletion | | |
| | | Donor | | | |
| rs9333341 | TSPO | Exon 3 Coding | C > G | Leu44Val | 22 |
| | | Sequence | | | |
| rs6971 | TSPO | Exon 4 Coding | C > T^{a} | Ala147Thr | 22 |
| | | Sequence | | | |
| rs6972 | TSPO | Exon 4 Coding | G > A | Arg162His | 22 |
| | | Sequence | | | |
| rs8192467 | TSPO | Exon 4 Coding | G > T | Arg166Leu | 22 |
| | | Sequence | | | |
| rs9333342 | TSPO | Exon 4 Coding | C > G | Glu169Gln | 22 |
| | | Sequence Splice | | | |
| | | Donor | | | |
| rs6892975 | VDAC1 | Intron 1 Splicing Lariat | G > T | | 5 |
| rs11537680 | VDAC2 | Exon 5 Coding | C > T | Thr66Ile | 10 |
| | | Sequence | | | |
| rs60564032 | VDAC2 | Exon 7 Coding | G > del | Asp188— | 10 |
| | | Sequence | | | |
| rs34070926 | VDAC2 | Exon 10 Coding | C > G | Gly255Ala | 10 |
| | | Sequence | | | |
| rs74146301 | VDAC2 | Intron 10 Splicing | A > G | | 10 |
| | | Lariat | | | |
| rs62508038 | VDAC3 | Exon 6 Coding | C > G | Thr51Arg | 8 |
| | | Sequence | | | |
| rs34738318 | VDAC3 | Intron 6 Splicing Lariat | C > del | | 8 |
| rs35475165 | VDAC3 | Intron 6 Splicing Lariat | A > del | | 8 |
| rs3736649 | VDAC3 | Intron 7 Splicing Lariat | A > G | | 8 |
| rs11558228 | VDAC3 | Exon 8 Coding | G > T | Gly148Trp | 8 |
| | | Sequence | | | |

| | | | | Single | |
|---|---|---|---|---|---|
| | | | | Marker | |
| | | | | Association | HWE |
| Polymorphism | NCBI build 35 | Genotyping | | Test p- | p- |
| ID | Position (bp) | Technology | Monomorphic? | value | value |
| rs3211100 | 224401051 | FAST | Yes | | |
| rs17855715 | 224401103 | FAST | Yes | | |
| rs1219666 | 224401201 | FAST | Yes | | |
| rs9730340 | 224416004 | FAST | Yes | | |
| rs2306120 | 224419121 | FAST | No | 0.466 | 1.000 |
| rs2301868 | 53737476 | FAST | No | 0.572 | 1.000 |
| rs9893754 | 53737494 | FAST | Yes | | |
| rs3744103 | 53738052 | FAST | No | 0.749 | 1.000 |
| rs34184983 | 53738700 | FRET | Yes | | |
| rs2877876 | 53739347 | FRET | Yes | | |
| rs11652816 | 53739373 | FAST | No | 0.836 | 0.647 |
| rs72839988 | 53740144 | FAST | No | 0.749 | 1.000 |
| rs34937759 | 53740354 | FRET | Yes | | |
| rs61732758 | 53740996 | Sequencing | Yes | | |
| rs61754146 | 53741556 | FAST | Yes | | |
| rs61741210 | 53741731 | FAST | No | 0.605 | 1.000 |
| rs3744101 | 53742461 | FAST | Yes | | |
| rs34126526 | 53743146 | FRET | Yes | | |
| rs2680704 | 53743237 | FAST | No | 0.836 | 0.647 |
| rs3744099 | 53743302 | FAST | No | 1.000 | 1.000 |
| rs9674726 | 53743625 | FAST | Yes | | |
| rs2072144 | 53743926 | FAST | No | 0.938 | 0.645 |
| rs9913145 | 53744731 | FAST | No | 0.775 | 1.000 |
| rs9783836 | 53745132 | FAST | Yes | | |
| rs2072143 | 53748547 | FAST | No | 0.719 | 1.000 |
| rs2072147 | 53750756 | FAST | No | 0.572 | 1.000 |
| rs5821222 | 53750776 | FRET | Yes | | |
| rs11652447 | 53751602 | FAST | Yes | | |
| rs2072145 | 53751608 | FAST | No | 0.572 | 1.000 |
| rs61747227 | 53752924 | FAST | Yes | | |
| rs9894786 | 53755452 | FAST | Yes | | |
| rs1042560 | 186302972 | FAST | Yes | | |
| rs28999114 | 186303111 | FAST | Yes | | |
| rs62619215 | 186303241 | FRET | Yes | | |
| rs62620230 | 186303246 | Sequencing | Yes | | |
| rs55820580 | 186304014 | FAST | Yes | | |
| rs113193775 | 186304048 | FAST | Yes | | |
| rs111811353 | 186304049 | FAST | Yes | | |
| rs5996279 | 41876677 | FAST | No | 1.000 | 1.000 |
| rs9333313 | 41877316 | FRET | No | 1.000 | 1.000 |
| rs3937387 | 41885138 | FAST | No | 0.046 | 1.000 |
| rs34936584 | 41886995 | FRET | Yes | | |
| rs71758389 | 41887140 | PCR & | Yes | | |
| | | agarose gel | | | |
| | | electrophoresis | | | |
| rs9333341 | 41888842 | FAST | Yes | | |
| rs6971 | 41888870 | FRET | No | 3.12E−13 | 0.623 |
| rs6972 | 41888916 | FRET | No | 0.122 | 1.000 |
| rs8192467 | 41888928 | FRET | Yes | | |
| rs9333342 | 41888936 | FRET | Yes | | |
| rs6892975 | 133356632 | FAST | Yes | | |
| rs11537680 | 76648873 | FAST | Yes | | |
| rs60564032 | 76650712 | Sequencing | Yes | | |
| rs34070926 | 76659436 | FAST | Yes | | |
| rs74146301 | 76660652 | FAST | No | 1.000 | 0.084 |
| rs62508038 | 42375421 | FAST | Yes | | |
| rs34738318 | 42376301 | FRET | Yes | | |
| rs35475165 | 42376311 | Sequencing | Yes | | |
| rs3736649 | 42378456 | FAST | No | 1.000 | 1.000 |
| rs11558228 | 42378581 | FAST | Yes | | |

^{a}rs6971 SNP genotyping data is reflective of the antisense strand for both alleles. The alteration to the coding sequence is actually GCG [Ala] to ACG [Thr] as described in dbSNP (http://www.ncbi.nlm.nih.gov/projects/SNP/)
nd = not determined

### Data Analysis

### Platelet Binding Affinity

Binding data were analysed using GraphPad Prism 5.0. Single site and two site competition models were fitted using the least squares algorithm and model selection was compared using an F-test. The null hypothesis, that the data fitted a single site model, was rejected if the p value was less than 0.05. A K_{d} for [³H]PK11195 of 29.25 nM (6) was used to generate the Kᵢ for PBR28. HABs and LABs were defined as subjects with a single binding site with Kᵢ<15 nM or >100 nM respectively. MABs were defined as subjects with two binding sites. Data are expressed as the mean±standard error of the mean (SEM).

### Genetic Polymorphisms

The PBR28 ligand binding classification endpoint was analyzed using two grouping scenarios: LABs & MABs vs HABs and LABs vs MABs & HABs; or using three groups. Single marker analysis was performed by Fisher's exact test using SAS 9.2 (Cary, N.C., USA). The two-tailed p-value and estimated odds ratio with 95% confidence interval (CI) was calculated. A 5% significance threshold was used by applying a Bonferroni correction for the number of markers analyzed (n=20) (significance threshold p=0.0025). Linkage disequilibrium analysis (8) and departure from Hardy Weinberg Equilibrium (HWE) (9) was tested on data from Caucasian subjects. See Supplementary Methods for more detail.

This study was approved by North West London REC 1, reference number 10/H0722/38.

### Genotyping

The polymorphisms were genotyped variously using fluorescence resonance energy transfer (FRET)-based TaqMan® (according to manufacturer's instructions (AppliedBiosystems, CA, USA)), Luminex-based Flow Assorted SNP Typing (FAST) (Taylor et al., 2001), direct sequencing or polymerase chain reaction (PCR) fragment analysis. The genotyping platform used to assay each polymorphism can be found in Supplementary Table 1. The assay details for the rs6971 TaqMan® assay are described below. Details for other assays are available upon request.

The accuracy and robustness of the genotyping assays and the minor allele frequencies for each polymorphism were determined by generating data from 267 HapMap samples from different ethnic groups with previously published data (http://hapmap.ncbi.nlm.nih.gov/), and comparing genotype concordance between our data and published genotype data for these polymorphisms and subjects, where available.

rs6971TaqMan® Assay

### Reagents

The TaqMan® assay for rs6971 (C_{—}2512465_{—}20; part no. 4351379) and 2× TaqMan Genotyping mix were obtained from Applied Biosystems (CA, USA).

### PCR Amplification & Assay Analysis

PCR reactions were performed in a 96-well microtiter-plate on a GeneAmp PCR System 9700 (Applied Biosystems, CA, USA). The reaction mixture of 10 μl contained 20 ng genomic DNA, 3.5 μl of 2× TaqMan Genotyping Master Mix, 0.25 μl of the primers and probes mix purchased from Applied Biosystems (primers 900 nM; probe 200 nM) and 6.25 μl of double distilled water. The reactions were incubated for 2 min. at 50° C. and 10 min. at 95° C., followed by 50 cycles at 92° C. for 15 sec. and 60° C. for 1.5 min. After PCR amplification, endpoint plate read and allele calling was performed using an ABI 7900 HT (Applied Biosystems, CA, USA) and the corresponding SDS software (v2.2.2). The data was subsequently exported to Spotfire® software (TIBCO, Somerville, Mass.) to check calcd alleles.

### Statistical Analysis of Genetic Polymorphisms

For the statistical analysis of genetic polymorphisms, both additive and dominant genetic models were tested. To decide which set of covariates (age, sex or race) to include in the analysis, Firth's penalized logistic regression (17) was performed with stepwise procedure. No interaction (p≦0.05) was observed between the effect of the ligand binding status and any of the covariates. Departure from Hardy Weinberg Equilibrium (HWE) was tested using an exact test by considering the distribution of genotypes conditional on observed allele frequencies (9). In order to assist in interpretation of significant associations, linkage disequilibrium (LD) analysis was conducted to measure the association between alleles at different loci utilising the pairwise r²measure (8).

### Results

### Binding Class

Protein in platelets from 27/41 subjects (66%) showed ligand binding to a single class of high affinity sites (Kᵢ=2.17±0.17 nM). These subjects were classified as HABs. In 12/41 subjects (29%), the data fitted best to a two-site model with affinities of 2.23±0.31 nM and 297±43.3 nM. These subjects were classified as MABs. In the remaining 2/41 subjects (5%), the ligand bound to a single class of low affinity sites (Kᵢ=187±19.7 nM). These subjects were classified as LABs (FIG. 1a).

### Genetic Polymorphisms

Single marker analysis revealed that only one polymorphism (rs6971) surpassed the multiple testing p-value threshold of 0.0025, with the most significant p-value obtained for the LABs & MABs vs. HABs binary analysis, using either of the genetic models tested (FIG. 2a, Table 2). This polymorphism is located in exon 4 of the TSPO gene and is predicted to result in a non-conservative amino acid substitution at position 147 from alanine to threonine (Ala147Thr) in the 5^{th} transmembrane domain of the TSPO protein. Complete agreement was observed between the PBR28 binding phenotype for individual subjects and their rs6971 genotype (adjusted p-value 5.68×10⁻¹⁰; Table 4, FIG. 1b, for analysis with two groups; and p-value 3.1×10⁻¹³ for analysis with three groups). A linkage disequilibrium (LD) plot for the 5 TSPO markers analysed indicated that the low level of LD (FIG. 2b) could account for the weak, statistically non-significant associations observed for flanking TSPO polymorphisms, rs3937387 and rs6972 (FIG. 2a). This pattern of association is what would be expected for a co-dominant monogenic trait.

TABLE 4
TSPO rs6971 (Ala147Thr) genotype in study subjects versus ligand-
binding phenotype

| TSPO genotype | | | | |
|---|---|---|---|---|
| DNA | | Binding phenotype | | |
| (polymorphism | Protein | (subject, n) | | |
| rs6971) | (position 147) | HAB | MAB | LAB |
| C/C | Ala/Ala | 27 | | |
| C/T | Ala/Thr | | 12 | |
| T/T | Thr/Thr | | | 2 |

Ala = alanine,
Thr = threonine;
HAB = high affinity,
MAB = mixed affinity,
LAB = low affinity binders.
Distribution of rs6971 genotypes against ligand binding classification. Genotypes correspond to carriage of the 147 amino acid as follows: CC = Ala147/Ala147; CT = Ala147/Thr147; TT = Thr147/Thr147.

### Discussion

Here we demonstrate complete agreement between TSPO binding affinity class measured in human platelets with PBR28, and variation at a common polymorphism (rs6971) in the TSPO gene which leads to an amino acid substitution (Ala147Thr). These data indicate that variation in binding affinity of PBR28 for human platelets is a co-dominant monogenic trait.

This finding is highly significant for the interpretation of PET studies using [¹¹C]PBR28. We have not formally demonstrated concordance in binding class between platelets and brain or other organs, but agreement seems highly likely as PET data with [¹¹C]PBR28 suggests that the LAB phenotype appears consistent across all tissues within the same subject (4). We therefore believe that PBR28 binding affinity class in the brain (or other tissues) can be predicted simply by genotyping the TSPO rs6971 polymorphism. In the absence of an available TSPO radioligand which binds with equivalent affinity in all subjects and has a high signal to noise ratio, genotyping the TSPO rs6971 polymorphism will enable confident, quantitative comparisons of PET data between groups of subjects, either by ensuring all study participants are from the same binding class or by correcting PET data based on binding class.

Our results also have broader implications for PET studies using [¹⁸F]PBR06, [¹¹C]DAA1106, [¹¹C]DPA713, [¹⁸F]PBR111 and [¹¹C]AC-5216 because, although there is no data confirming that these ligands bind at the same site as PBR28, we have previously demonstrated consistency of binding class across different subjects for all of these radioligands (5). The results also could help to better understand pharmacokinetic-pharmacodynamic relationships for drugs targeting TSPO, as we have suggested previously based on data from direct binding affinity assays with XBD173 (AC-5216)(10).

This binding affinity variation has greatest impact for studies of Caucasians, for whom the rs6971 polymorphism has a reported minor allele (Thr147) frequency of 30% and a major allele (Ala147) frequency of 70% (11). The minor allele is considerably less prevalent in other populations, such as Han Chinese (2%), Japanese (4%), and African American (25%) (12). In our small predominantly Caucasian sample, the observed percentage of MABs and LABs (29% and 5% respectively) was lower than expected (42% and 9%, respectively) based on published frequencies, although they are not outside the 95% confidence bounds for sampling variation. This discrepancy could be explained by some subjects inaccurately reporting their own ancestral background and/or the result of an unknown bias in ascertainment.

Identification of the functional effects of the rs6792 polymorphism prompted us to explore structural modelling of the protein using a general platform in wide use (PolyPhen software) (13). This suggested that substitution of threonine. (neutral and polar) for alanine (neutral and hydrophobic) at position 147 of TSPO could alter the protein tertiary structure (PolyPhen score 0.999, data not shown). Alanine 147 is highly conserved across most species (14), and likely contributes to maintaining the helical structure of the 5^{th} transmembrane domain of the protein. Protein structure data based on mouse and bacterial TSPO suggests that this helical conformation could play a key role in TSPO function (14), (15). We therefore hypothesise that the Ala147Thr amino acid substitution results in a conformational change affecting the interaction between TSPO and the variety of molecules for which affinity differences have been demonstrated (5), (6), (10).

There is some evidence suggesting that the Ala147Thr substitution has an impact on biological functions of TSPO. An association between the polymorphism and variation in pregnenolone production and plasma levels of LDL-cholesterol has been reported in healthy individuals (11). A small pilot study in patients with a diagnosis of depression also found an association between the polymorphism and separation anxiety (16). However, neither of these findings has been replicated yet.

While the relatively small size of our sample is a potential limitation of this study, the perfect concordance between binding affinity class and the rs6791 polymorphism is striking. Direct testing of the relationships between genetic variation, platelet binding and [¹¹C]PBR28 PET signal in vivo will further confirm the link.

### Conclusion

The relative affinity of the PET radioligand [¹¹C]PBR28 for TSPO in human platelets is determined by a single polymorphism (rs6791) in the TSPO gene. Our results therefore indicate that a simple test of genotype will enable determination of TSPO ligand binding class to allow quantitative assessments of TSPO density using PET.

### EXAMPLE 2

### Overview of TSPO Radioligands for PET

There are two main classes in the series of molecules reported for use in humans thus far (n=7). One for which the lead molecule is DAA1106 comprising: PBR28, PBR06, FBR and also Emapunil. The second family is lead by PK11195, a bi-cyclic “linker”, which includes also DPA713 and PBR111. Note that number of molecules is limited and actually PK11195 alternatively could be structurally distinguished from PBR111 and DPA713.

For the “High Affinity Receptor”, we have not identified a strong trend in structure-binding relations. For the “Low Affinity receptor” we have identified 3 distinct affinities: lower than 31 nM (DAA1106, PK11195 and Emapunil), around 60 nM (PBR111 and DPA713) and higher than 100 nM (PBR06 and PBR28), but we have not identified a strong trend in structure-binding relations since the structurally closely related modules DAA1106 and PBR28/PBR06 display 2 different behaviours.

The chemical structure may not be a direct predictor of the TSPO binding class, although there is a crude trend for the bi-cyclic linker family (PK11195 family: no or limited differential binding). Binding affinities and structures are summarised below.

| | Ki HAB | Ki MAB | Ki LAB | LAB/HAB |
|---|---|---|---|---|
| DAA1106 | 2.8 ± 0.3 | 4.8 ± 0.4 | 13.1 ± 1.3 | 4.7 |
| PK11195 | 28.3 ± 4 | 23.6 ± 5.5 | 22.3 ± 2.2 | 0.8 |
| Emapunil | 2.4 ± 0.67 | 10.5 ± 0.99 | 30.3 ± 8.7 | 12.6 |
| PBR111 | 15.6 ± 3.7 | 30.3 ± 4.1 | 61.8 ± 10.7 | 4 |
| DPA713 | 15 ± 2.2 | 26.8 ± 2.9 | 66.4 ± 7.8 | 4.4 |
| PBR06 | 8.6 ± 2.0 | 13.4 and 176 | 149 ± 46.6 | 17 |
| PBR28 | 3.4 ± 0.2 | 4.0 and 313 | 188 ± 7.0 | 55 |
| GE1 | 12.35 ± 2.1 | | 145.11 ± 22.1 | 11.8 |
| (racemate) | | | | |
| See | | | | |
| Example 3 | | | | |

Ki's were determined using a method as set out in J Nucl Med. 2011 January; 52(1):24-32) which describes the binding studies in human brain from which this data comes.

### Reference List

(1) Venneti S, Lopresti B J, Wiley C A. The peripheral benzodiazepine receptor (Translocator protein 18 kDa) in microglia: from pathology to imaging. Prog Neurobiol. 2006; 80(6):308-322.
(2) Banati R B, Newcombe J, Gunn R N et al. The peripheral benzodiazepine binding site in the brain in multiple sclerosis: quantitative in vivo imaging of microglia as a measure of disease activity. Brain. 2000;123 (Pt 11):2321-2337.
(3) Chauveau F, Boutin H, Van C N, Dolle F, Tavitian B. Nuclear imaging of neuroinflammation: a comprehensive review of [11C]PK11195 challengers. Eur J Nucl Med Mol Imaging. 2008; 35(12):2304-2319.
(4) Kreisl W C, Fujita M, Fujimura Y et al. Comparison of [(11)C]-(R)-PK 11195 and [(11)C]PBR28, two radioligands for translocator protein (18 kDa) in human and monkey: Implications for positron emission tomographic imaging of this inflammation biomarker. Neuroimage. 2010; 49(4):2924-2932.
(5) Owen D R, Gunn R N, Rabiner E A et al. Mixed-affinity binding in humans with 18-kDa translocator protein ligands. J Nucl Med. 2011; 52(1):24-32.
(6) Owen D R, Howell O W, Tang S P et al. Two binding sites for [(3)H]PBR28 in human brain: implications for TSPO PET imaging of neuroinflammation. J Cereb Blood Flow Metab. 2010.
(7) Taylor J D, Briley D, Nguyen Q et al. Flow cytometric platform for high-throughput single nucleotide polymorphism analysis. Biotechniques. 2001; 30(3):661-669.
(8) VanLiere J M, Rosenberg N A. Mathematical properties of the r2 measure of linkage disequilibrium. Theor Popul Biol. 2008; 74(1):130-137.
(9) Wigginton J E, Cutler D J, Abecasis G R. A note on exact tests of Hardy-Weinberg equilibrium. Am J Hum Genet. 2005; 76(5):887-893.
(10) Owen D R, Lewis A J, Reynolds R et al. Variation in binding affinity of the novel anxiolytic XBD173 for the 18 kDa translocator protein in human brain. Synapse. 2011; 65(3):257-259.
(11) Costa B, Pini S, Gabelloni P et al. The spontaneous Ala147Thr amino acid substitution within the translocator protein influences pregnenolone production in lymphomonocytes of healthy individuals. Endocrinology. 2009; 150(12):5438-5445.
(12) http://hapmap.ncbi.nlm.nih.gov/cgi-per/snp_details_phase3?name=rs6971&source=hapmap28_B36&tmpl=snp_details_phase3
(13) Ramensky V, Bork P, Sunyaev S. Human non-synonymous SNPs: server and survey. Nucleic Acids Res. 2002; 30(17):3894-3900.
(14) Murail S, Robert J C, Coic Y M et al. Secondary and tertiary structures of the transmembrane domains of the translocator protein TSPO determined by NMR. Stabilization of the TSPO tertiary fold upon ligand binding. Biochim Biophys Acta. 2008; 1778(6):1375-1381.
(15) Korkhov V M, Sachse C, Short J M, Tate C G. Three-dimensional structure of TspO by electron cryomicroscopy of helical crystals. Structure. 2010; 18(6):677-687.
(16) Costa B, Pini S, Martini C et al. Ala147Thr substitution in translocator protein is associated with adult separation anxiety in patients with depression. Psychiatr Genet. 2009; 19(2):110-111.
(17) Firth D. Bias reduction of maximum likelihood estimates Biometrika. 1993; 80(1):27-38.
(18) Barrett J C, Fry B, Mailer J, Daly M J. Haploview: analysis and visualization of LD and haplotype maps. Bioinformatics. 2005; 21(2):263-265.

### EXAMPLE 3

### Analysis of the Active Enantiomer Compound Described in US 201110070161.

This compound was prepared substantially as described in US 2011/0070161, except that the sequence was changed so that the alkylation with fluoroethyltosylate was done on the tricyclic intermediate, in order to improve the yield.

The synthesised compound was analysed by NMT and LC-MS and the expected results were obtained.

Binding assays were performed as for the other compounds, as described above. The compound concentrations used were 10000 nM, 3000 nM. 1000 nM, 300 nM, 100 nM. 30 nM, 10 nM, 3 nM, 1 nM, 0.3 nM and 0.03 nM. 6 HABS and 6 LABs were used.

### Results:

The Ki values averaged 12.35 nM for HABs and 145.11 nM for LABs (11.8 fold difference). All data fit best to a model which assumed just one binding site (with one exception, patient 200, further discussion below). As the racemate was used in this experiment, it can only be concluded that the average affinity shift for both enantiomers is approximately 12. For example, (S)GE1 could have an affinity shift of 13 fold and (R)GE1 could have an affinity shift of 11 fold. If this were the case, then the LAB affinity of (S)GE1 would be ˜160 nM and of (R)GE1 would be ˜135 nM. These two affinities would not be detected within LABs because they are so close. However, it cannot be argued that (S)GE1 has no shift at all and (R)GE1 has a shift of 24 fold. Were this the case then in the LAB assays, two binding sites would be detected (Ki=12 nM for (S)GE1 and Ki=290 nM for (R)GE1) as they would differ by a large enough margin to be mathematically resolved.

Re: patient 200. With all subjects we have compared two models (one model assuming one binding site and one model assuming two sites) to see which fits the data better. We use a p value of 0.05 to compare, and so if 20 assays are performed (on subjects with a single site) it is expected that 1 will erroneously appear to express two sites through the play of chance (eg noise in the data). Indeed, the software user manual stresses to interpret these tests with caution, within the known biological context. In this instance, the model suggests that pt 200 has two binding sites (with affinities 0.1 nM and 398 nM, distributed in the ratio 25%/75%). This would be a highly unusual result with no clear explanation. Therefore, given the p value with pt 200 was 0.049 (ie only just favouring the hypothesis that there were two sites), and as all other LABs clearly fit a one site model, it highly likely that this is a false positive and in fact the single site model is more appropriate.

GE1

| | | [3H- | PK11195 | |
|---|---|---|---|---|
| | IC 50 | PK11195] (nM) | Kd (nM) | Ki (nM) |
| HABs | | | | |
| A | 16.11 | 5 | 29.25 | 13.76 |
| 16 | 10.68 | 5 | 29.25 | 9.12 |
| 22 | 14.9 | 5 | 29.25 | 12.72 |
| 32 | 21.23 | 5 | 29.25 | 18.13 |
| 313 | 19.31 | 5 | 29.25 | 16.49 |
| 330 | 4.51 | 5 | 29.25 | 3.85 |
| | | | MEAN | 12.35 |
| | | | SD | 5.202657 |
| | | | SEM | 2.1 |
| LABs | | | | |
| 26 | 102.7 | 5 | 29.25 | 87.71 |
| 36 | 107.6 | 5 | 29.25 | 91.89 |
| 37 | 161.5 | 5 | 29.25 | 137.92 |
| 74 | 135.1 | 5 | 29.25 | 115.38 |
| 200 | 275.5 | 5 | 29.25 | 235.28 |
| 203 | 169.9 | 5 | 29.25 | 145.10 |
| | | | MEAN | 145.11 |
| | | | SD | 54.12012 |
| | | | SEM | 22.1 |
| | | | Affinity | 11.8 |
| | | | shift | |

### EXAMPLE 4

### Vinpocetine—a TSPO Radioligand Selective for the Low Affinity Binding Site

The 18 kDa Translocator Protein (TSPO) has been proposed as a marker of activated microglia. A single nucleotide polymorphism in the gene encoding the TSPO (rs6971) leads to higher affinity in AA subjects (high affinity binders) compared with TT subjects (low affinity binders) for the majority of TSPO ligands. Here, we provide an example of a compound with higher affinity for the TT phenotype compared with the AA phenotype. This impacts on recent PET studies with ¹¹C-vinpocetine and raises the possibility that, in low affinity binders, vinpocetine may cause sufficient occupancy to have pharmacodynamic effects with therapeutic value.

### Introduction

Quantitative imaging of TSPO with positron emission tomography (PET) has been technically challenging. The poor signal-to-noise ratio (SNR) and difficulties in deriving an arterial plasma input function for the first generation ligand [¹¹C]PK11195 limits its utility. Several second generation TSPO ligands with improved SNR, including [¹¹C]PBR28, [¹⁸F]PBR06, [¹⁸F]FEPPA, [¹¹C]DAA1106, [¹¹C]DPA713 and [¹⁸F]PBR111 have been investigated in man. We have recently shown that binding affinity of these radioligands is affected by a polymorphism (rs6971) in the gene encoding the TSPO (Owen et al. 2011; Owen et al. 2010; Owen et al. 2012b; preceding Examples). This polymorphism causes a single amino acid substitution of alanine (A) for threonine (T) at position 147 in the TSPO. Subjects with the TT phenotype bind examples of TSPO-targeting radioligands with a reduced affinity relative to AA subjects, and have hence been labelled low and high affinity binders (LAB and HAB) respectively. The heterozygotes (AT) express both versions of the protein in similar proportions, display an intermediate affinity for the TSPO ligands and have been labelled mixed affinity binders (MAB). The presence of differing affinities in the general population complicates the quantitative assessment of PET data, because differences in signal cannot be safely interpreted as differences in target density. The genotyping of participants in PET studies for the TSPO rs6971 polymorphism allows the determination of binding affinity class, and allows the quantitative comparisons of PET data between groups of patients.

Vinpocetine is a synthetic compound derived from the plant Vinca minor which has recently been radiolabelled and used as TSPO targeting PET ligand (Gulyas et al. 2011). Whilst its reported binding affinity (200 nM in the rat heart (Gulyas et al. 2005)) is an order of magnitude lower than other TSPO targeting ligands, this is counter balanced by high brain penetration which may allow for sufficient specific binding to produce a measurable specific PET signal (Vas and Gulyas 2005).

As well as its recent use in PET, vinpocetine has been trialled as a therapeutic agent for various diseases including cerebrovascular disease, cognitive impairment and epilepsy (Patyar et al. 2011). Efficacy has not been convincingly demonstrated in any condition and hence the drug is not licenced in the UK or USA. These studies have been based on the premise of proposed independent interactions of vinpocetine with sodium channels, calcium channels, phosphodiesterases or NF-κB. However, the affinity of vinpocetine for these targets is low (Kᵢ in the uM range) compared with the affinity for TSPO derived from the rat heart (Bonoczk et al. 2000; Chiu et al. 1988; Hagiwara et al. 1984; Jeon et al. 2010; Souness et al. 1989; Tretter and Adam-Vizi 1998).

The binding of vinpocetine for the TSPO in humans has only been investigated quantitatively in one previous study (Hall et al. 2002). In this [¹¹C]autoradiography study, binding affinity was not formally measured but a high affinity interaction (<100 nM) was excluded. We investigated the binding affinity of vinpocetine for TSPO in the human brain in post mortem samples, using donors from HABs and LABs.

### Materials and Methods

### Brain Tissue

Tissue was obtained from the UK Multiple Sclerosis (MS) Tissue Bank at Imperial College. All tissue blocks obtained included only “normal appearing” tissue, without immunohistochemical evidence of demyelination or significant inflammatory infiltrate. The tissue was stored at −80° C. until use. Tissue blocks from 15 donors (5 each from HAB, MAB and LAB donors) were used. The binding affinity classification for each tissue block had been established previously using PBR28 (Owen et al. 2010).

### Materials

[³H]PK11195(1-(2-Chlorophenyl)-N-methyl-N-(1-methylpropyl)-3-isoquinolinecarboxamide specific activity=80 Ci/mmol; radioactive concentration=1.0 mCi/ml) was purchased from Perkin Elmer, UK. Unlabelled PK11195 and vinpocetine was purchased from Sigma, UK.

### Membrane Preparation

Tissue blocks were homogenised in 10 times weight for volume (w/v) buffer (0.32 mM sucrose, 5 mM Tris base, 1 mM MgCl₂, pH 7.4, 4° C.). Homogenates were centrifuged (32,000 g, 20 min, 4° C.) followed by removal of the supernatant. Pellets were re-suspended in at least 10 times w/v buffer (50 mM Tris base, 1 mM MgCl₂, pH 7.4, 4° C.) followed by two washes by centrifugation (32,000 g, 20 min, 4° C.). Membranes were suspended in buffer (50 mM Tris base, 1 mM MgCl₂, pH 7.4, 4° C.) at a protein concentration of approximately 4 mg protein/ml and aliquots were stored at −80° C. until use.

### Competition Binding Assays

Aliquots (approximately 250 μg protein/ml) of membrane suspension were prepared using assay buffer (50 mM Tris base, 140 mM NaCl, 1.5 mM MgCl₂, 5 mM KCl, 1.5 mM CaCl₂, pH 7.4, 37° C.) and incubated with [³H]PK11195 (5 nM) and one of 10 concentration of vinpocetine, ranging from 0.3 nM to 10 μM, in a final volume of 500 μl for 60 min at 37° C. The specific binding component was determined using unlabelled PK11195 (10 μM). Following incubation, assays were terminated via filtration through Whatman GF/B filters, followed by 3×1 ml washes with ice-cold wash buffer (50 mM Tris Base, 1.4 mM MgCl₂, pH 7.4, 4° C.). Whatman GF/B filters were pre-incubated with 0.05% polyethyleneimine (60 min) before filtration. Scintillation fluid (4 ml/vial, Perkin Elmer Ultima Gold MV) was added and vials counted on a Perkin Elmer Tricarb 2900 liquid scintillation counter. For each donor, each point was performed in quadruplicate. Kᵢ (nM) values were determined using GraphPad Prism 5.0 software (GraphPad Software Inc, USA).

### Protein Concentration Determination

Protein concentrations (μg protein/ml) were determined using the Bicinchoninic acid assay (BCA Kit, Sigma-Aldrich, UK) and absorption read at 562 nm.

### Data Analysis

Data were analysed using the iterative non-linear regression curve fitting software supplied with GraphPad Prism 5.0. Single site and two site competition models were fitted to the data using the least squares algorithm and model selection was performed using an F-test. The null hypothesis, that the data fitted a single site model, was rejected if the p value was less than 0.05. A K_{d} for [³H]PK11195 of 29.25 nM (Owen et al. 2010) was used to generate the Kᵢ for vinpocetine according to the Cheng and Prusoff equation. Data are expressed as the mean (standard error of the mean). ANOVA with a p value was less than 0.05 (GraphPad Prism 5.0) was used to determine statistical significance.

### Results

Data from all subjects (including MABs) were best described by a single site model. The mean Kᵢ values of vinpocetine for HAB (AA subjects) was 777.3 (74.8) nM, for LAB (TT subjects) was 109.1 (3.2) nM and for MAB (AT subjects) was 331.6 (76.3) nM (FIG. 1, table 1, p<0.0001). The Kᵢ value for MAB subjects (331.6 nM) was similar to the mean Kᵢ value of HABs and LABs (443.2 nM).

### Discussion

With the exception of PK11195, whose binding to the TSPO appears unaffected by the rs6971 polymorphism, all previous TSPO drug ligands and PET radioligands investigated to date bind with a higher affinity in AA subjects (HAB) relative to TT subjects (LAB). Vinpocetine is the first ligand identified which binds with highest affinity in TT subjects, ie low affinity binders. AT subjects (MAB), who express both binding sites, appear to express a single class of binding sites with an affinity similar to the mean of the HAB and LAB sites. This inability to resolve the two binding sites in MABs has been found with all TSPO ligands where the difference in affinity between HABs and LABs is less than 10 fold (Owen et al. 2011).

This finding has important implications for PET studies with ¹¹C-vinpocetine, as between-subject comparisons in the specific signal derived from these studies will be invalid unless subjects' binding affinity class is determined and corrected for. This can be achieved either by excluding subjects at screening to ensure all study participants are from the same binding class, or by including all subjects but subsequently correcting PET data based on binding class.

These data also have potential consequences for the use of vinpocetine as a therapeutic. Although it has been trialled on the basis of phosphodiesterase inhibition and blockade of sodium and calcium channels, the concentrations required for these effects (1-44 uM) are 1-2 orders of magnitude higher than the K, value for TSPO in TT subjects (˜100 nM). Therefore any pharmacodynamic effects seen with vinpocetine are likely to be a result of interaction with the TSPO, rather than other targets. The differences in affinity seen in the binding of vinpocetine indicate that clinical studies designed to test the therapeutic effects of vinpocetine should be conducted in genetically homogenous population, and the TT phenotype which constitutes ˜10% of the Caucasian population is most likely to demonstrate therapeutic effects. The ambiguous results seen in previous clinical studies may be a consequence of differences in binding affinity class and hence vinpocetine occupancy of the TSPO.

The data presented here raises the possibility of whether, in TT and possibly AT subjects, sufficient occupancy of the TSPO can be achieved clinically (with a pharmacologically appropriate dose of vinpocetine) to produce a pharmacodynamic effect and modulate TSPO function. Studies measuring occupancy of unlabelled vinpocetine and testing TSPO mediated pharmacodynamic endpoints are underway to test this hypothesis. These data also add emphasis to the need for genotyping subjects in clinical trials to avoid missing drug effect because a proportion of the target population does not bind the drug (Owen et al. 2012a).

TABLE 5

| Amino acid at position 147 | | |
|---|---|---|
| of TSPO | Binding affinity class | Vinpocetine Kᵢ |
| A/A | High affinity binder | 777 (74.8) nM |
| T/T | Low affinity binder | 109 (3.2) nM |
| A/T | Mixed affinity binder | 332 (76.3) nM |

A = alanine,
T = threonine.

### Reference List

Bonoczk P, Gulyas B, Adam-Vizi V, Nemes A, Karpati E, Kiss B, Kapas M, Szantay C, Koncz I, Zelles T, Vas A (2000) Role of sodium channel inhibition in neuroprotection: effect of vinpocetine. Brain Res Bull 53:245-254
Chiu P J, Tetzloff G, Ahn H S, Sybertz E J (1988) Comparative effects of vinpocetine and 8-Br-cyclic GMP on the contraction and 45Ca-fluxes in the rabbit aorta. Am J Hypertens 1:262-268
Gulyas B, Halldin C, Vas A. Banati R B, Shchukin E, Finnema S, Tarkainen J, Tihanyi K, Szilagyi G, Farde L (2005) [11C]vinpocetine: a prospective peripheral benzodiazepine receptor ligand for primate PET studies. J Neurol Sci 229-230:219-223
Gulyas B, Vas A, Toth M, Takano A, Varrone A, Cselenyi Z, Schain M, Mattsson P, Halldin C (2011) Age and disease related changes in the translocator protein (TSPO) system in the human brain: positron emission tomography measurements with [11C]vinpocetine. Neuroimage 56:1111-1121
Hagiwara M, Endo T, Hidaka H (1984) Effects of vinpocetine on cyclic nucleotide metabolism in vascular smooth muscle. Biochem Pharmacol 33:453-457
Hall H, Vamas K, Sandell J, Halldin C, Farde L, Vas A, Karpati E, Gulyas B (2002) Autoradiographic evaluation of [11C]vinpocetine binding in the human postmortem brain. Acta Biol Hung 53:59-66
Jeon K I, Xu X, Aizawa T, Lim J H, Jono H, Kwon D S, Abe J, Berk B C, Li J D, Yan C (2010) Vinpocetine inhibits NF-kappaB-dependent inflammation via an IKK-dependent but PDE-independent mechanism. Proc Natl Acad Sci USA 107:9795-9800
Owen D R, Gunn R N, Rabiner E A, Bennacef I, Fujita M, Kreisl W C, Innis R B, Pike V W, Reynolds R, Matthews P M, Parker C A (2011) Mixed-affinity binding in humans with 18-kDa translocator protein ligands. J Nucl Med 52:24-32
Owen D R, Howell O W, Tang S P, Wells L A, Bennacef I, Bergstrom M, Gunn R N, Rabiner E A, Wilkins M R, Reynolds R, Matthews P M, Parker C A (2010) Two binding sites for [(3)H]PBR28 in human brain: implications for TSPO PET imaging of neuroinflammation. J Cereb Blood Flow Metab
Owen D R, Rupprecht R, Nutt D J (2012a) Stratified medicine in psychiatry: a worrying example or new opportunity in the treatment of anxiety? J Psychopharmacol
Owen D R, Yeo A J, Gunn R N, Song K, Wadsworth G, Lewis A, Rhodes C, Pulford D J, Bennacef I, Parker C A, StJean P L, Cardon L R, Mooser V E, Matthews P M, Rabiner E A, Rubio J P (2012b) An 18-kDa translocator protein (TSPO) polymorphism explains differences in binding affinity of the PET radioligand PBR28. J Cereb Blood Flow Metab 32:1-5
Patyar S, Prakash A, Modi M, Medhi B (2011) Role of vinpocetine in cerebrovascular diseases. Pharmacol Rep 63:618-628
Souness J E, Brazdil R, Diocee B K, Jordan R (1989) Role of selective cyclic GMP phosphodiesterase inhibition in the myorelaxant actions of M&B 22,948, MY-5445, vinpocetine and 1-methyl-3-isobutyl-8-(methylamino)xanthine. Br J Pharmacol 98:725-734
Tretter L, Adam-Vizi V (1998) The neuroprotective drug vinpocetine prevents veratridine-induced [Na+]i and [Ca2+]i rise in synaptosomes. Neuroreport 9:1849-1853
Vas A, Gulyas B (2005) Eburnamine derivatives and the brain. Med Res Rev 25:737-757

1. A method for aiding in selecting or adjusting a dose of TSPO imaging or therapeutic agent for use with a subject, the method comprising the step of determining the subject's genotype for TSPO rs6971.

2-3. (canceled)

4. The method of claim 1 wherein the agent is an agent that binds with differential affinity to Ala147TSPO (encoded by the allele in which C is present at polymorphism rs6971) and Thr147TSPO (encoded by the allele in which T is present at polymorphism rs6971).

5. The method of claim 4 wherein the agent binds with higher affinity to Ala 147TSPO than to Thr147TSP and when the subject's genotype for TSPO rs6971 is determined to be CC the dose of TSPO imaging or therapeutic agent is reduced.

6. A method for TSPO imaging or therapy wherein the subject's genotype at TSPO polymorphism position rs6971 is determined and wherein either (i) the subject's genotype for TSPO is taken into account when assessing the results of the TSPO imaging or (ii) the subject's genotype for TSPO is taken into account when choosing the TSPO agent and/or the dose of TSPO agent.

7-8. (canceled)

9. The method of claim 6 wherein the subject's determined measure of binding of the TSPO agent is normalised to take account of the subject's determined genotype.

10. (canceled)

11. The method of claim 6 wherein the subject is a subject who has been determined to have genotype CC or CT at TSPO polymorphism position rs69715 when using a TSPO agent that binds with higher affinity to Ala147TSPO than to Thr147TSPO.

12. The method of claim 6 wherein the subject is a subject who has been determined to have genotype TT or CT at TSPO polymorphism position rs6971 when using a TSPO agent that binds with higher affinity to Thr147TSPO than to Ala147TSPO.

13. A method of aiding in determining whether a subject is suitable for TSPO imaging or therapy using a TSPO imaging or therapeutic agent, the method comprising the step of selecting the subject as suitable for TSPO imaging or therapy based on the subject's genotype at TSPO polymorphism position rs6971.

14-15. (canceled)

16. A kit of parts comprising a TSPO imaging or therapeutic agent and a reagent specifically for assessing a subject's genotype at TSPO rs6971.

17. The method of claim 4 wherein the TSPO imaging or therapeutic agent that binds with differential affinity to Ala 147TPSO and Thr147TSPO has a Ki for the lower affinity binding that is at least three-fold higher than the Ki for the higher affinity binding.

18. The method of claim 1 wherein the TSPO imaging agent is PBR28; PBR06; DAA1106; AC-5216; PBR111; DPA713; FEPPA; IGA-1; or (4S)—N,N-diethyl-9-(2-fluoroethyl)-5-methoxy-2,3,4,4a,9,9a-hexahydro-1H-carbazole-4-carboamide.

19. The method of claim 1 wherein the TSPO therapeutic agent is Emapunil/XBD173 or vinpocetine.

20. (canceled)

21. The method of claim 6 wherein the TSPO imaging agent is PBR28; PBR06; DAA1106; AC-5216; PBR111; DPA713; FEPPA; IGA-1; or (4S)—N,N-diethyl-9-(2-fluoroethyl)-5-methoxy-2,3,4,4a,9,9a-hexahydro-1H-carbazole-4-carboamide.

22. The method of claim 6 wherein the TSPO therapeutic agent is Emapunil/XBD173 or vinpocetine.

23. The method of claim 13 wherein the TSPO imaging agent is PBR28; PBR06; DAA1106; AC-5216; PBR111; DPA713; FEPPA; IGA-1; or (4S)—N,N-diethyl-9-(2-fluoroethyl)-5-methoxy-2,3,4,4a,9,9a-hexahydro-1H-carbazole-4-carboamide.

24. The method of claim 13 wherein the TSPO therapeutic agent is Emapunil/XBD173 or vinpocetine.

25. The kit of claim 16 wherein the TSPO imaging agent is PBR28; PBR06; DAA1106; AC-5216; PBR111; DPA713; FEPPA; IGA-1; or (4S)—N,N-diethyl-9-(2-fluoroethyl)-5-methoxy-2,3,4,4a,9,9a-hexahydro-1H-carbazole-4-carboamide.

26. The kit of claim 16 wherein the TSPO therapeutic agent is Emapunil/XBD173 or vinpocetine.


SEQUENCE LISTING (3 sequences)

SEQ ID NO: 1 - DNA, 12 bp, Homo sapiens
ggtcgctgaagg

SEQ ID NO: 2 - DNA, 22 bp, Artificial Sequence
  Forward primer to determine genotype at TSPO rs6971
tgggacaggcacttgggtgaac

SEQ ID NO: 3 - DNA, 22 bp, Artificial Sequence
  Reverse primer to determine genotype at TSPO rs6971
aagcgtgacggccaccacatca